(19) United States
(12) Patent Application Publication
(10) Pub. No.: US 2025/0263361 A1
(43) Pub. Date: Aug. 21, 2025

(54) Process For the Production of Dimethyl Ether and Hydrogen from Methane Using a Solid Metal Oxide Reagent

(71) Applicants: TOTALENERGIES ONETECH, Courbevoie (FR); SULZER MANAGEMENT AG, Winterthur (CH)

(72) Inventors: Gleb VERYASOV, Seneffe (BE); Nikolai NESTERENKO, Winterthur (CH); Zhongyi DING, Katy, TX (US); Mircea-Gheorghe CRETOIU, Sugar Land, TX (US); Miguel Alexandre FIGUEIREDO SANTOS, Sugar Land, TX (US); Jingsong ZHOU, Houston, TX (US)

(21) Appl. No.: 18/994,829

(22) PCT Filed: Jul. 24, 2023
(86) PCT No.: PCT/EP2023/070453

§ 371 Date: Jan. 15, 2025

(30) Foreign Application Priority Data
Jul. 25, 2022 (EP) 22315166.3
Jul. 25, 2022 (EP) 22315167.1
Jul. 25, 2022 (EP) 22315168.9

(51) Int. Cl. C07C 41/01; B01D 53/14; C01B 7/09; C07C 41/34; C25B 1/02

(52) U.S. Cl. CPC C07C 41/01; B01D 53/1456; C01B 7/093; C07C 41/34; B01D 2257/2042; C25B 1/02

(57) ABSTRACT

The present invention relates to a process for producing dimethyl ether (DME) and hydrogen (H₂) from methane, comprising the steps of: a) providing a gaseous feed stream comprising methane; b) reacting said gaseous feed stream with at least one halogen reactant (X₂), under reaction conditions effective to produce an effluent stream comprising methyl halide (MeX), and hydrogen halide (HX); c) separating from the effluent stream obtained in step b): (i) a methyl halide (MeX) stream; and, (ii) a hydrogen halide (HX) stream; d) reacting the methyl halide (MeX) stream separated in step c) with a solid metal oxide (MO₍ₛ₎) under reaction conditions effective to produce metal halide (MX) and dimethyl ether (DME); and e) decomposing by means of electrolysis said hydrogen halide (HX) stream separated in step c) under conditions effective to produce a gaseous hydrogen (H₂) stream and a stream comprising halogen reactant (X₂).

### TECHNICAL FIELD

The present invention in general relates to a process and a system for producing dimethyl ether. In particular, the present invention relates to a process and system for the production of dimethyl ether and hydrogen from a gas stream comprising or consisting of methane. The present process is in particular characterised in that it utilizes a solid metal oxide reactant to form dimethyl ether, and in that it uses electrolysis to produce hydrogen gas.

### BACKGROUND

Dimethyl ether (DME, CH₃OCH₃), is a colourless, nontoxic, non-corrosive, non-carcinogenic, and environmentally friendly chemical. DME has a boiling point of −25° C., and is a gas under ambient conditions. DME is, however, easily handled as liquid and stored in pressurized tanks, much like liquefied petroleum gas (LPG).

There are a few number of applications for DME such as aerosol, propellant, chemical feedstock, transportation fuel, or refrigerant. For instance, today DME is mainly used as an aerosol propellant in spray cans, in place of the banned CFC gases.

Another major application of DME is its use as feedstock for the synthesis of other products, such as dimethyl sulphate, methyl acetate, and light olefins. DME can also be converted into acetic acid through carbonylation.

DME can also be used as a direct and cleaner fuel alternative to conventional diesel. In addition, DME is also an important chemical intermediate for the production of widely used chemicals, such as diethyl sulphate, methyl acetate, or olefins. DME is considered an ultra-clean transportation fuel because it has a high cetane number (around 60), low boiling point (−25° C.), and high oxygen content (35 wt %) which allow fast vaporization and higher combustion quality (smokeless operation and 90% less NOx emissions) than other alternative CO₂-based fuels.

DME is usually produced via two main ways: the first is the indirect route where DME is obtained by the dehydration of methanol and the second is the direct route where DME is produced directly from syngas (hydrogen and carbon monoxide) in a single stage thanks to bi-functional catalysts.

In the indirect route, a first step involves the catalytic hydrogenation of syngas into methanol (CO/CO₂+3H₂→CH₃OH+H₂O) and a second step involves the subsequent catalytic dehydration of the methanol over acid catalysts to produce DME (₂CH₃OH→CH₃OCH₃+H₂O). The main problem of this two-step DME synthesis derives from the strong thermodynamic limitation of the methanol synthesis step that leads to low gas conversion per pass (15-25%) and therefore high recirculation ratios and high capital and operating costs. Moreover, processes for synthesizing methanol from syngas typically entrain CO and CO₂, as well as many other light and heavy weight coproducts along with the methanol product. The coproducts are a result of the more complex series of reactions that take place when the three reactant gasses interact with each other and the catalytic surface. Much of the subsequent energy and cost in conventional methanol production plants is directed to these coproducts since they must be separated from the methanol product.

In the early 1990s researchers began to study the direct synthesis of DME from syngas in a process in which methanol synthesis step is coupled in situ with the dehydration step in a single reactor (2CO/CO₂+6H₂→CH₃OCH₃+3H₂O). The direct synthesis of DME allows for increasing the level of conversion per step, up to 90%, which means significant savings in capital investment and production costs of DME. In fact, it is estimated that DME production costs in direct synthesis are reduced by 20-30% compared to the traditional two-step process.

However, direct DME synthesis from syngas requires highly efficient hybrid bifunctional catalytic systems which should combine a CO/CO₂ hydrogenation function for methanol synthesis and an acidic function for methanol dehydration. Moreover, the production of syngas itself often involves cracking of natural gas, which is very energy consuming as the rection is endothermic and is typically performed at high temperatures.

The document US2002/0198416 discloses a process wherein methane reacts with bromine to form CH₃Br and HBr. These components then react with a metal oxide to form a mixture of DME and metal bromide. In a first manner, CH₃Br and HBr react in a converter with a metal oxide to form DME. In a second manner, the HBr first reacts with the metal oxide to form a metal bromide and water. The metal bromide is then regenerated into the metal oxide in a regenerator. Then, water, CH₃Br, and CH₄ are sent into a separator where after separation, CH₃Br is sent to a reactor to react with the regenerated metal oxide to generate DME.

Hence, in view of the above, there is a need in the art to provide improved processes for producing DME. There is in particular a demand in the art for providing processes for producing DME having improved efficiencies. There is also a demand in the art for DME production processes that can operate under milder production process conditions, avoiding working at highly elevated temperatures and/or pressures that require excessive amounts of energy to maintain these temperatures and/or pressures.

There is also a demand in the art for DME production processes that involve lower CO/CO₂ emissions.

There is a further demand in the art for DME production processes allowing to produce DME via a different strategy, especially not starting from a syngas route.

It is, therefore, an object of the present invention to provide a process for producing DME which allows fulfilling at least some of the above-indicated demands.

### SUMMARY OF THE INVENTION

In accordance with the present invention, an improved process for producing dimethyl ether and hydrogen gas from methane is provided.

In a first aspect, the present invention thereto provides a process for producing dimethyl ether (DME) and hydrogen (H₂) from methane, comprising the steps of:
a) providing a gaseous feed stream comprising methane;
b) reacting said gaseous feed stream with at least one halogen reactant (X₂), under reaction conditions effective to produce an effluent stream comprising methyl halide (MeX), and hydrogen halide (HX), optionally polyhalogenated alkanes, and optionally unreacted methane;
c) separating from the effluent stream obtained in step b):
(i) a methyl halide (MeX) stream, optionally comprising unreacted methane; and,
(ii) a hydrogen halide (HX) stream;
d) reacting the methyl halide (MeX) stream separated in step c) with a solid metal oxide (MO₍ₛ₎), under reaction conditions effective to produce metal halide (MX) and dimethyl ether (DME); and,
e) decomposing by means of electrolysis said hydrogen halide (HX) stream separated in step c) under conditions effective to produce a gaseous hydrogen (H₂) stream and a stream comprising halogen reactant (X₂).

In particular, the present invention thereto provides a process for producing dimethyl ether (DME) and hydrogen (H₂) from methane, comprising the steps of:
a) providing a gaseous feed stream comprising methane;
b) reacting said gaseous feed stream with at least one halogen reactant (X₂), under reaction conditions effective to produce an effluent stream comprising methyl halide (MeX), and hydrogen halide (HX), optionally polyhalogenated alkanes, and optionally unreacted methane;
c) separating from the effluent stream obtained in step b):
   (i) a methyl halide (MeX) stream, optionally comprising unreacted methane; and,
   (ii) an aqueous hydrogen halide (HX) stream or a hydrogen halide (HX) stream;
d) reacting the methyl halide (MeX) stream separated in step c) with a solid metal oxide (MO₍ₛ₎), under reaction conditions effective to produce metal halide (MX) and dimethyl ether (DME); and,
e) decomposing by means of electrolysis said aqueous hydrogen halide (HX) stream or said hydrogen halide (HX) stream separated in step c) under conditions effective to produce a gaseous hydrogen (H₂) stream and a stream comprising halogen reactant (X₂);
wherein in step c) the hydrogen halide (HX) stream is separated from the effluent stream by contacting the effluent stream comprising said hydrogen halide with an aqueous absorbent, or by distillation, thereby forming respectively an aqueous hydrogen halide (HX) stream or a hydrogen halide (HX) stream.

In some embodiments, the process further comprises the step of separating said polyhalogenated alkanes formed in step b) from said effluent stream, preferably prior to separating said methyl halide (MeX) stream and said hydrogen halide stream from said effluent stream.

In certain embodiments, the present invention provides a process for producing dimethyl ether (DME) and hydrogen (H₂) from methane, comprising the steps of:
a) providing a gaseous feed stream comprising methane;
b) reacting said gaseous feed stream with at least one halogen reactant (X₂), under reaction conditions effective to produce an effluent stream comprising methyl halide (MeX), and hydrogen halide (HX), polyhalogenated alkanes, and optionally unreacted methane;
c) separating from the effluent stream obtained in step b):
   (i) a methyl halide (MeX) stream, optionally comprising unreacted methane; and,
   (ii) a hydrogen halide (HX) stream; and
   (iii) a polyhalogenated alkanes stream;
d) reacting the methyl halide (MeX) stream separated in step c) with a solid metal oxide (MO₍ₛ₎), under reaction conditions effective to produce metal halide (MX) and dimethyl ether (DME); and,
e) decomposing by means of electrolysis said hydrogen halide (HX) stream separated in step c) under conditions effective to produce a gaseous hydrogen (H₂) stream and a stream comprising halogen reactant (X₂).

In some embodiments, said polyhalogenated alkanes comprise polyhalogenated methane and optionally polyhalogenated C₂₊ alkanes.

In some embodiments, said halogen reactant is selected from the group consisting of bromine (Br₂), chlorine (Cl₂), fluorine (F₂), iodine (I₂), and astatine (At₂), and preferably is bromine (Br₂).

In some embodiments, in step c) the hydrogen halide (HX) stream is separated from the effluent stream by contacting the effluent stream comprising said hydrogen halide with an aqueous absorbent, preferably an aqueous hydrogen halide (HX) solution, thereby forming an aqueous hydrogen halide (HX) stream.

In some embodiments, C₂₊ alkyl monohalides are formed during step b).

In some embodiments, the process comprises the step of separating C₂₊ alkyl monohalides formed in step b) of the process from said effluent stream, and preferably the process comprises separating said C₂₊ alkyl monohalides formed in step b) from said effluent stream prior to separating said methyl halide (MeX) stream and said hydrogen halide stream from said effluent stream.

In some embodiments, the gaseous feed stream comprises at least 75.0 vol % methane, and preferably at least 80.0 vol % methane, preferably at least 85.0 vol % methane, preferably at least 90.0 vol % methane, preferably at least 95.0 vol % methane, preferably at least 98.0 vol % methane, preferably at least 99.0 vol % methane, preferably at least 99.9 vol % methane, based on the total volume of the gaseous feed stream.

In some embodiments, the gaseous feed stream applied in step a) of the process is substantially free of sulphur species, e.g. it contains less than 1.0 mol %, or less than 0.5 mol %, or less than 0.1 mol %, or less than 0.01 mol %, or less than 0.001 mol %, or less than 0.0001 mol %, or less than 0.00001 mol % of sulphur species.

In some embodiments, the solid metal oxide (MO)₍ₛ₎) is a metal oxide provided on a solid support, preferably wherein said support is a silica or an alumina support.

In some embodiments, the process further comprises the step of:
f) regenerating the metal halide (MX) to metal oxide (MO) and halogen reactant (X₂), and preferably by contacting the metal halide (MX) with oxygen (O₂) or air, thereby forming metal oxide (MO) and halogen reactant (X₂).

In some embodiments, the process further comprising the step of returning the halogen reactant (X₂) obtained in step e) and/or step f), to step b) of the process.

In some embodiments, the gaseous hydrogen (H₂) stream generated in step e) comprises at least 90.0 mol %, such as at least 95.0 mol %, or at least 99.0 mol %, or at least 99.5 mol %, or at least 99.9 mol % hydrogen.

In some embodiments, the process further comprises the steps of:
removing, preferably distilling, unreacted methane, from the dimethyl ether produced in step d), and
optionally, recycling or feeding said removed unreacted methane into step a) and/or step b) of the process.

In a second aspect, the present invention provides a system for producing dimethyl ether (DME) and hydrogen (H₂) from methane, comprising:
a halogenation reactor (1), configured to react a gaseous feed stream comprising methane with at least one halogen reactant (X₂) into an effluent stream comprising methyl halide (MeX), and hydrogen halide (HX), and optionally unreacted methane,
a separation system (2), which is fluidly connected to said halogenation reactor (1) and configured to receive an effluent stream from said halogenation reactor (1) and configured to separate a hydrogen halide (HX) stream and a methyl halide (MeX) stream from said effluent stream;
a dimethyl ether synthesis reactor (3), which is fluidly connected to said separation system (2), and configured to receive a methyl halide (MeX) stream from said separation system (2) and to react said methyl halide (MeX) stream with a solid metal oxide (MO₍ₛ₎) into metal halide (MX) and dimethyl ether (DME); and
an electrolysis unit (4) comprising at least one electrolysis cell and a power source for supplying current to said electrolysis cell, which is fluidly connected to said separation system (2), and configured to receive a hydrogen halide (HX) stream and to decompose said hydrogen halide (HX) stream into a gaseous hydrogen (H₂) stream and a stream comprising halogen reactant (X₂);
a feed stream supply system (5) for supplying a gaseous feed stream comprising methane to said halogenation reactor (1);
a halogen supply system (6) for supplying a halogen reactant (X₂) to said halogenation reactor (1);
an effluent recovery system (10), configured to recover an effluent stream from said halogenation reactor (1), and for feeding said recovered effluent stream to said separation system (2);
a dimethyl ether recovery system (7), configured to recover a stream comprising dimethyl ether (DME) from said dimethyl ether synthesis reactor (3);
a hydrogen recovery system (8), configured to recover a gaseous hydrogen (H₂) stream from said electrolysis unit (4);
a halogen recovery system (9), configured to recover halogen reactant (X₂) from said electrolysis unit (4);
a hydrogen halide transfer system (12), configured to recover a hydrogen halide (HX) stream from said separation system (2), and to supply said recovered hydrogen halide (HX) stream to the electrolysis unit (4);
a methyl halide transfer system (11), configured to recover a methyl halide (MeX) stream from said separation system (2), and to supply said recovered methyl halide (MeX) stream to the dimethyl ether synthesis reactor (3),
optionally, an absorbent supply line (17), for supplying an aqueous absorbent, preferably an aqueous hydrogen halide (HX) solution, to said separation system (2);
optionally, a polyhalogenated alkanes removal unit, configured to receive effluent stream from said halogenation reactor (1) and to remove polyhalogenated alkanes from said effluent stream, and,
optionally, a C₂₊ alkyl monohalide removal unit, configured to receive effluent stream from said halogenation reactor (1) and to remove C₂₊ alkyl monohalides from said effluent stream.

The present process advantageously allows to produce a substantially pure dimethyl ether stream, e.g. which is essentially free of methanol (MeOH), and preferably other alcohols and/or ethers. The process of the invention avoids any direct CO₂ emissions and shows improved selectivity for the production of dimethyl ether. In addition, the present process also allows to obtain a substantially pure stream of hydrogen gas as co-product, and which can be further used in numerous downstream applications.

The present invention also provides a process for producing dimethyl ether in which no syngas is involved. In accordance with the present invention, dimethyl ether, and hydrogen are directly produced starting from methane without the need to produce syngas. The methane used in the process can come from a renewable source, which makes the production of the dimethyl ether and the base chemicals made from said dimethyl ether, more eco-friendly.

The present process is also highly efficient as it allows to recycle (re-use) reagents and reaction products in the same process, thereby reducing waste and/or pollution.

Moreover, the process allows to produce hydrogen gas by the decomposition of a hydrogen halide stream by means of electrolysis. Hence, at least part of the energy applied in the present process is based on electric energy, which can be obtained from a renewable source.

The independent and dependent claims set out particular and preferred features of the invention. Features from the dependent claims may be combined with features of the independent or other dependent claims as appropriate.

The present invention will now be further described. In the following passages, different aspects of the invention are defined in more detail. Each aspect so defined may be combined with any other aspect or aspects unless clearly indicated to the contrary. In particular, any feature indicated as being preferred or advantageous may be combined with any other feature or features indicated as being preferred or advantageous.

### DETAILED DESCRIPTION OF THE FIGURES

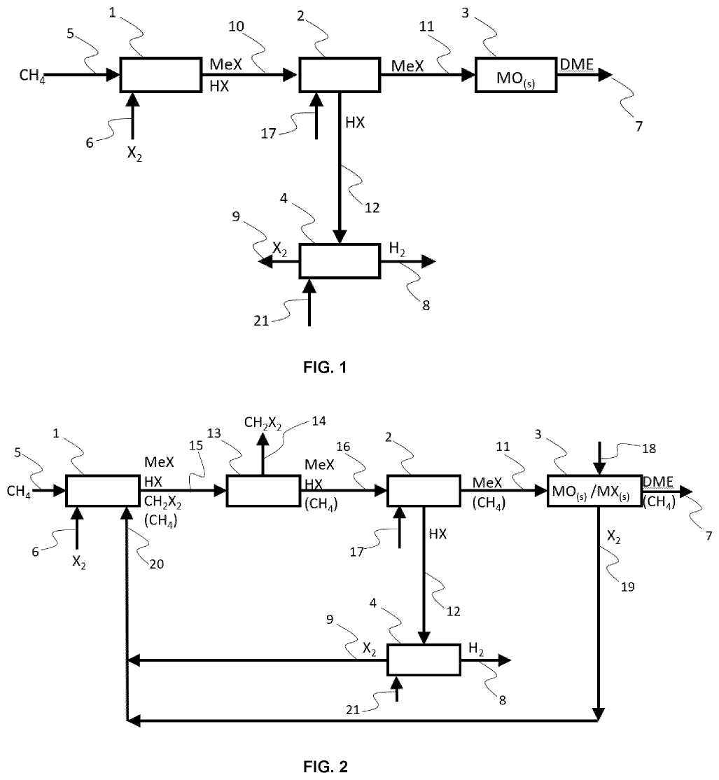

FIG. 1 depicts a process according to an embodiment of the invention, and a system according to an embodiment of the invention.

FIG. 2 depicts a process according to another embodiment of the invention, and a system according to an embodiment of the invention.

### DETAILED DESCRIPTION OF THE INVENTION

When describing the invention, the terms used are to be construed in accordance with the following definitions, unless a context dictates otherwise.

Unless otherwise defined, all terms used in disclosing the invention, including technical and scientific terms, have the meaning as commonly understood by one of ordinary skill in the art to which this invention belongs. By means of further guidance, term definitions are included to better appreciate the teaching of the present invention.

In the following passages, different aspects of the invention are defined in more detail. Each aspect so defined may be combined with any other aspect or aspects unless clearly indicated to the contrary. In particular, any feature indicated as being preferred or advantageous may be combined with any other feature or features indicated as being preferred or advantageous.

Reference throughout this specification to “one embodiment” or “an embodiment” means that a particular feature, structure, or characteristic described in connection with the embodiment is included in at least one embodiment of the present invention. Thus, appearances of the phrases “in one embodiment” or “in an embodiment” in various places throughout this specification are not necessarily all referring to the same embodiment, but may. Furthermore, the particular features, structures, or characteristics may be combined in any suitable manner, as would be apparent to a person skilled in the art from this disclosure, in one or more embodiments. Furthermore, while some embodiments described herein include some but not other features included in other embodiments, combinations of features of different embodiments are meant to be within the scope of the invention, and form different embodiments, as would be understood by those in the art.

The terms “comprising”, “comprises” and “comprised of” as used herein are synonymous with “including”, “includes” or “containing”, “contains”, and are inclusive or open-ended and do not exclude additional, non-recited members, elements, or process steps. It will be appreciated that the terms “comprising”, “comprises” and “comprised of” as used herein comprise the terms “consisting of”, “consists” and “consists of”.

As used in the specification and the appended claims, the singular forms “a”, “an,” and “the” include plural referents unless the context clearly dictates otherwise. By way of example, “a step” means one step or more than one step.

Unless defined otherwise, all technical and scientific terms used herein have the same meaning as is commonly understood by one of skill in the art.

The recitation of numerical ranges by endpoints includes all integer numbers and, where appropriate, fractions subsumed within that range (e.g. 1 to 5 can include 1, 2, 3, 4 when referring to, for example, a number of elements, and can also include 1.5, 2, 2.75 and 3.80, when referring to, for example, measurements). The recitation of endpoints also includes the end point values themselves (e.g. from 1.0 to 5.0 includes both 1.0 and 5.0). Any numerical range recited herein is intended to include all sub-ranges subsumed therein.

The term “about” as used herein when referring to a measurable value such as a parameter, an amount, a temporal duration, and the like, is meant to encompass variations of +/−10% or less, preferably +/−5% or less, more preferably +/−1% or less, of and from the specified value, insofar such variations are appropriate to perform in the disclosed invention. It is to be understood that the value to which the modifier “about” refers is itself also specifically, and preferably, disclosed.

The terms “wt %”, “vol %”, or “mol %” refers to a weight percentage of a component, a volume percentage of a component, or molar percentage of a component, respectively, based on the total weight, the total volume of material, or total moles, that includes the component.

When describing the present invention, the terms used are to be construed in accordance with the following definitions, unless a context dictates otherwise.

Whenever the term “substituted” is used in the present invention, it is meant to indicate that one or more hydrogens on the atom indicated in the expression using “substituted” is replaced with a selection from the indicated group, provided that the indicated atom's normal valency is not exceeded and that the substitution results in a chemically stable compound, i.e. a compound that is sufficiently robust to survive isolation to a useful degree of purity from a reaction mixture.

The term “alkyl” as a group or part of a group, refers to a hydrocarbyl group of formula CₙH₂ₙ₊₁ wherein n is a number greater than or equal to 1. Alkyl groups may be linear or branched and may be substituted as indicated herein. Generally, alkyl groups of this invention comprise from 1 to 20 carbon atoms, preferably from 1 to 18 carbon atoms, preferably from 1 to 12 carbon atoms, preferably from 1 to 10 carbon atoms, preferably from 1 to 6 carbon atoms, more preferably 1, 2, 3, 4, 5, 6 carbon atoms. When a subscript is used herein following a carbon atom, the subscript refers to the number of carbon atoms that the named group may contain. For example, the term “C₁₋₂₀alkyl”, as a group or part of a group, refers to a hydrocarbyl group of formula —CₙH₂ₙ₊₁ wherein n is a number ranging from 1 to 20. Thus, for example, C₁₋₂₀alkyl groups include all linear, or branched alkyl groups having 1 to 20 carbon atoms, and thus includes for example methyl, ethyl, n-propyl, i-propyl, 2-methyl-ethyl, butyl and its isomers (e.g. n-butyl, i-butyl and t-butyl); pentyl and its isomers, hexyl and its isomers, heptyl and its isomers, octyl and its isomers, nonyl and its isomers, decyl and its isomers, undecyl and its isomers, dodecyl and its isomers, tridecyl and its isomers, tetradecyl and its isomers, pentadecyl and its isomers, hexadecyl and its isomers, heptadecyl and its isomers, octadecyl and its isomers, and the like. For example, C₁₋₁₀alkyl includes all linear, or branched alkyl groups having 1 to 10 carbon atoms, and thus includes for example methyl, ethyl, n-propyl, i-propyl, 2-methyl-ethyl, butyl, and its isomers (e.g. n-butyl, i-butyl, and t-butyl); pentyl and its isomers, hexyl and its isomers, heptyl and its isomers, octyl and its isomers, nonyl and its isomers, decyl and its isomers and the like. For example, C₁₋₆alkyl includes all linear, or branched alkyl groups having 1 to 6 carbon atoms, and thus includes for example methyl, ethyl, n-propyl, i-propyl, 2-methyl-ethyl, butyl and its isomers (e.g. n-butyl, i-butyl, and t-butyl); pentyl and its isomers, hexyl and its isomers. In some embodiments, non-limiting examples of alkyl groups include for instance methyl, ethyl, propyl, iso-propyl, n-butyl, isobutyl, sec-butyl, t-butyl, pentyl, isopentyl, 2,2-dimethyl-propyl, hexyl, 2,3-dimethyl-2-butyl, heptyl, 2,2-dimethyl-3-pentyl, 2-methyl-2-hexyl, octyl, 4-methyl-3-heptyl, nonyl, decyl, undecyl and dodecyl groups.

As used herein and unless otherwise stated, the term “halo” or “halogen”, as a group or part of a group, is generic for any atom selected from the group consisting of fluorine (F), chlorine (CI), bromine (Br), iodine (I) and astatine (At).

Preferred statements (features) and embodiments and uses of this invention are set herein below. Each statement and embodiment of the invention so defined may be combined with any other statement and/or embodiment unless clearly indicated to the contrary. In particular, any feature indicated as being preferred or advantageous may be combined with any other feature or features or statements indicated as being preferred or advantageous. Hereto, the present invention is in particular captured by any one or any combination of one or more of the below-numbered statements and embodiments, with any other aspect and/or embodiment.

1. A process for producing dimethyl ether (DME) and hydrogen (H₂) from methane, comprising the steps of:
a) providing a gaseous feed stream comprising methane;
b) reacting said gaseous feed stream with at least one halogen reactant (X₂), under reaction conditions effective to produce an effluent stream comprising methyl halide (MeX), and hydrogen halide (HX), optionally polyhalogenated alkanes, and optionally unreacted methane;
c) separating from the effluent stream obtained in step b):
   (i) a methyl halide (MeX) stream, optionally comprising unreacted methane; and,
   (ii) a hydrogen halide (HX) stream;
d) reacting the methyl halide (MeX) stream separated in step c) with a solid metal oxide (MO₍ₛ₎), under reaction conditions effective to produce metal halide (MX) and dimethyl ether (DME); and,
e) decomposing by means of electrolysis said hydrogen halide (HX) stream separated in step c) under conditions effective to produce a gaseous hydrogen (H₂) stream and a stream comprising halogen reactant (X₂).
   2. The process according to statement 1, wherein the process comprises the step of recovering a stream comprising dimethyl ether and optionally unreacted methane after step d).
   3. The process according to statement 1 or 2, wherein the process further comprises the step of separating said polyhalogenated alkanes formed in step b) from said effluent stream, preferably prior to separating said methyl halide (MeX) stream and said hydrogen halide stream from said effluent stream.
   4. A process, preferably a process according to any one of statements 1 to 3, for producing dimethyl ether (DME) and hydrogen (H₂) from methane, comprising the steps of:
a) providing a gaseous feed stream comprising methane;
b) reacting said gaseous feed stream with at least one halogen reactant (X₂), under reaction conditions effective to produce an effluent stream comprising methyl halide (MeX), and hydrogen halide (HX), polyhalogenated alkanes, and optionally unreacted methane;
c) separating from the effluent stream obtained in step b):
   (i) a methyl halide (MeX) stream, optionally comprising unreacted methane; and,
   (ii) a hydrogen halide (HX) stream; and
   (iii) a polyhalogenated alkanes stream;
d) reacting the methyl halide (MeX) stream separated in step c) with a solid metal oxide (MO₍ₛ₎), under reaction conditions effective to produce metal halide (MX) and dimethyl ether (DME); and,
e) decomposing by means of electrolysis said hydrogen halide (HX) stream separated in step c) under conditions effective to produce a gaseous hydrogen (H₂) stream and a stream comprising halogen reactant (X₂).
   5. The process according to statement 4, wherein the process comprises the step of recovering a stream comprising dimethyl ether and optionally unreacted methane after step d).
   6. The process according to any one of statements 1 to 5, wherein said polyhalogenated alkanes comprise polyhalogenated methane and optionally polyhalogenated C₂+ alkanes.
   7. The process according to any one of statements 1 to 6, wherein said halogen reactant is selected from the group consisting of bromine (Br₂), chlorine (Cl₂), fluorine (F₂), iodine (I₂), and astatine (At₂).
   8. The process according to any one of the previous statements, wherein said halogen reactant is bromine (Br₂).
   9. The process according to any one of the previous statements, wherein steps b) and d) are performed in separate reaction steps, preferably in separate reactors or separate reaction zones.
   10. The process according to any one of the previous statements, wherein the temperature during step b) is at least 150° C., preferably at least 200° C., preferably at least 250° C., preferably at least 300° C., preferably at least 350° C., preferably at least 380° C.
   11. The process according to any one of the previous statements, wherein the temperature during step b) is at most 900° C., preferably at most 800° C., preferably at most 700° C., preferably at most 600° C., preferably at most 550° C., preferably at most 520° C.
   12. The process according to any one of the previous statements, wherein the temperature during step b) is at least 150° C. to at most 900° C., preferably at least 200° C. to at most 800° C., preferably at least 250° C. to at most 700° C., preferably at least 300° C. to at most 600° C., preferably at least 350° C. to at most 550° C., preferably at least 380° C. to at most 520° C.
   13. The process according to any one of the previous statements, wherein the pressure during step b) is at least 1.0 bar, preferably at least 1.5 bar, preferably at least 2.0 bar, preferably at least 2.5 bar, preferably at least 3.0 bar, preferably at least 5.0 bar, preferably at least 10.0 bar.
   14. The process according to any one of the previous statements, wherein the pressure during step b) is at most 50.0 bar, preferably at most 45.0 bar, preferably at most 40.0 bar, preferably at most 35.0 bar, preferably at most 30.0 bar, preferably at most 25.0 bar, preferably at most 20.0 bar.
   15. The process according to any one of the previous statements, wherein the pressure during step b) is at least 1.0 bar to at most 50.0 bar, preferably at least 1.5 bar to at most
   45.0 bar, preferably at least 2.0 bar to at most 40.0 bar, preferably at least 2.5 bar to at most 35.0 bar, preferably at least 3.0 bar to at most 30.0 bar, preferably at least 5.0 bar to at most 25.0 bar, preferably at least 10.0 bar to at most 20.0 bar.
   16. The process according to any one of the previous statements, wherein in step b) the gaseous feed stream is contacted with the halogen reactant (X₂) for at least 0.1 min to at most 2.0 min, preferably for at least 0.2 min to at most 1.7 min, preferably for at least 0.5 min to at most 1.5 min, preferably for at least 0.7 min to at most 1.2 min.
   17. The process according to any one of the previous statements, wherein in step c) the hydrogen halide (HX) stream is separated from the effluent stream by contacting the effluent stream comprising said hydrogen halide with an aqueous absorbent, capable of absorbing said hydrogen halide (HX), and preferably an aqueous hydrogen halide (HX) solution, thereby forming an aqueous hydrogen halide (HX) stream.
   18. The process according to any one of the previous statements, wherein polyhalogenated alkanes are formed during step b), preferably wherein the formed polyhalogenated alkanes formed during step b) comprise polyhalogenated methane and optionally polyhalogenated C₂₊ alkanes.
   19. The process according to any one of the previous statements, wherein polyhalogenated alkanes formed in step b) of the process comprise methylene dihalide (CH₂X₂).
   20. The process according to any one of the previous statements, wherein the process comprises the step of separating polyhalogenated alkanes formed in step b) from said effluent stream, thereby creating a polyhalogenated alkanes stream, and preferably the process comprises separating said polyhalogenated alkanes formed in step b) from said effluent stream prior to separating hydrogen halide from said effluent stream.
   21. The process according to any one of the previous statements, wherein polyhalogenated alkanes formed in step b) of the process are separated from the effluent stream by distillation.
   22. The process according to any one of the previous statements, wherein polyhalogenated alkanes formed in step b) of the process are separated from the effluent stream as a polyhalogenated methane stream and a polyhalogenated C₂₊ alkanes stream, preferably by distillation.
   23. The process according to any one of the previous statements, wherein C₂₊ alkyl monohalides are formed in step b).
   24. The process according to any one of the previous statements, wherein the process comprises the step of separating C₂₊ alkyl monohalides formed in step b) of the process from said effluent stream.
   25. The process according to any one of the previous statements, wherein the process comprises separating said C₂₊ alkyl monohalides formed in step b) from said effluent stream prior to separating hydrogen halide from said effluent stream.
   26. The process according to any one of the previous statements, wherein the process comprises separating said C₂₊ alkyl monohalides formed in step b) from said effluent stream after separating said polyhalogenated alkanes from said effluent stream.
   27. A process for producing dimethyl ether (DME) and hydrogen (H₂) from methane, preferably according to any one of the previous statements, comprising the steps of:
a) providing a gaseous feed stream comprising methane;
b) reacting said gaseous feed stream with at least one halogen reactant (X₂), under reaction conditions effective to produce an effluent stream comprising methyl halide (MeX), and hydrogen halide (HX), polyhalogenated alkanes, C₂₊ alkyl monohalides, and optionally unreacted methane;
c) separating from the effluent stream obtained in step b) the following substreams:
   c1) a polyhalogenated alkanes stream;
   c2) a C₂₊ alkyl monohalides stream;
   c3) a hydrogen halide (HX) stream; and
   c4) a methyl halide (MeX) stream, optionally comprising unreacted methane;
d) reacting the methyl halide (MeX) stream separated in step c) with a solid metal oxide (MO₍ₛ₎), under reaction conditions effective to produce metal halide (MX) and dimethyl ether (DME); and,
e) decomposing by means of electrolysis said hydrogen halide (HX) stream separated in step c) under conditions effective to produce a gaseous hydrogen (H₂) stream and a stream comprising halogen reactant (X₂).
   28. The process according to statement 27, wherein the process comprises the step of recovering a stream comprising dimethyl ether and optionally unreacted methane after step d).
   29. The process according to any one of the previous statements, wherein C₂₊ alkyl monohalides formed in step b) of the process are separated from the effluent stream by distillation.
   30. The process according to any one of the previous statements, wherein the methyl halide (MeX) stream separated in step c) is a gaseous stream.
   31. The process according to any one of the previous statements, wherein the methyl halide (MeX) stream separated in step c) is essentially free of polyhalogenated alkanes.
   32. The process according to any one of the previous statements, wherein the methyl halide (MeX) stream separated in step c) is essentially free of C₂₊ alkyl monohalides.
   33. The process according to any one of the previous statements, wherein the hydrogen halide (HX) stream obtained in step c) is dry.
   34. The process according to any one of the previous statements, wherein step c) comprises separating from the effluent stream:
(i) a methyl halide (MeX) stream, optionally comprising unreacted methane and optionally C₂₊ alkyl monohalides;
(ii) a hydrogen halide (HX) stream; and,
(iii) optionally a polyhalogenated alkanes stream, which may comprise a polyhalogenated methane stream and a polyhalogenated C₂₊ alkanes stream.
   35. The process according to any one of the previous statements, wherein the methyl halide (MeX) stream comprises unreacted methane.
   36. The process according to any one of the previous statements, wherein the methyl halide (MeX) stream comprises C₂₊ alkyl monohalides.
   37. The process according to any one of the previous statements, wherein step c) comprises separating from the effluent stream:
(i) a methyl halide (MeX) stream, optionally comprising unreacted methane;
(ii) a hydrogen halide (HX) stream; and,
(iii) a polyhalogenated alkanes stream, comprising polyhalogenated methane stream and optionally polyhalogenated C₂₊ alkanes stream; and,
(iv) optionally a C₂₊ alkyl monohalides stream.
   38. The process according to any one of the previous statements, wherein the gaseous feed stream comprises at least 75.0 vol % methane, preferably at least 80.0 vol % methane, preferably at least 85.0 vol % methane, preferably at least 90.0 vol % methane, preferably at least 95.0 vol % methane, preferably at least 98.0 vol % methane, preferably at least 99.0 vol % methane, preferably at least 99.9 vol % methane, based on the total volume of the gaseous feed stream.
   39. The process according to any one of the previous statements, wherein the gaseous feed stream comprises natural gas, a biogas, a refinery gas, or any mixture thereof.
   40. The process according to any one of the previous statements, wherein the gaseous feed stream applied in step a) of the process is substantially free of sulphur species, e.g. it contains less than 1.0 mol %, or less than 0.5 mol %, or less than 0.1 mol %, or less than 0.01 mol %, or less than 0.001 mol %, or less than 0.0001 mol %, or less than 0.00001 mol % of sulphur species.
   41. The process according to any one of the previous statements, wherein the solid metal oxide (MO)₍ₛ₎) is a metal oxide provided on a solid support, preferably wherein said support is a silica or an alumina support.
   42. The process according to any one of the previous statements, wherein the metal oxides selected from the list comprising or consisting of calcium oxide (CaO), magnesium oxide (MgO), barium oxide (BaO), copper oxide (CuO), and any mixtures thereof.
   43. The process according to any one of the previous statements, wherein the metal oxide is calcium oxide (CaO), magnesium oxide (MgO) or a mixture thereof, preferably the metal oxide is a mixture of calcium oxide (CaO) and magnesium oxide (MgO).
   44. The process according to any one of the previous statements, wherein in step d) the methyl halide (MeX) is contacted with the solid metal oxide (MO₍ₛ₎) for at least 0.1 min to at most 100.0 min, preferably for at least 0.2 min to at most 75.0 min, preferably for at least 0.5 min to at most 60.0 min, preferably for at least 1.0 min to at most 45.0 min, preferably for at least 2.0 min to at most 30.0 min, preferably for at least 3.0 min to at most 15.0 min, preferably for at least 4.0 min to at most 10.0 min, preferably for at least 4.0 min to at most 6.0 min.
   45. The process according to any one of the previous statements, wherein the temperature during step d) is at least 50° C., preferably at least 70° C., preferably at least 80° C., preferably at least 90° C., preferably at least 100° C., preferably at least 120° C.
   46. The process according to any one of the previous statements, wherein the temperature during step d) is at most 500° C., preferably at most 450° C., preferably at most 400° C., preferably at most 350° C., preferably at most 300° C., preferably at most 250° C.
   47. The process according to any one of the previous statements, wherein the temperature during step d) is at least 50° C. to at most 500° C., preferably at least 70° C. to at most 450° C., preferably at least 80° C. to at most 400° C., preferably at least 90° C. to at most 350° C., preferably at least 100° C. to at most 300° C., preferably at least 120° C. to at most 250° C.
   48. The process according to any one of the previous statements, wherein the pressure during step d) is at least 1.0 bar, preferably at least 1.2 bar, preferably at least 1.5 bar, preferably at least 1.7 bar, preferably at least 2.0 bar, preferably at least 5.0 bar, preferably at least 10.0 bar.
   49. The process according to any one of the previous statements, wherein the pressure during step d) is at most 40.0 bar, preferably at most 35.0 bar, preferably at most 30.0 bar, preferably at most 25.0 bar, preferably at most 20.0 bar, preferably at most 15.0 bar, preferably at most 10.0 bar.
   50. The process according to any one of the previous statements, wherein the pressure during step d) is at least 1.0 bar to at most 40.0 bar, preferably at least 1.2 bar to at most 35.0 bar, preferably at least 1.5 bar to at most 30.0 bar, preferably at least 1.7 bar to at most 25.0 bar, preferably at least 2.0 bar to at most 20.0 bar, preferably at least 5.0 bar to at most 20.0 bar, preferably at least 10.0 bar to at most 15.0 bar.
   51. The process according to any one of the previous statements, wherein polyhalogenated methanes are converted to methanol, preferably by hydrogenation of polyhalogenated methane to form methyl halide, followed by contacting methyl halide with a solid metal hydroxide (MOH₍ₛ₎), under reaction conditions effective to produce the corresponding methanol.
   52. The process according to any one of the previous statements, wherein polyhalogenated methanes are converted to DME, preferably by hydrogenation of polyhalogenated methane to form methyl halide, followed by contacting methyl halide with a solid metal oxide, under reaction conditions effective to produce DME.
   53. The process according to any one of the previous statements, wherein the process further comprises the step:
f) regenerating the metal halide (MX) to metal oxide (MO) and halogen reactant (X₂).
   54. The process according to any one of the previous statements, wherein the metal halide (MX) is regenerated in step f) by contacting the metal halide (MX) with oxygen (O₂) or air, thereby forming metal oxide (MO) and halogen reactant (X₂).
   55. The process according to any one of the previous statements, wherein the temperature during step f) is at least 150° C., preferably at least 200° C., preferably at least 250° C., preferably at least 300° C., preferably at least 350° C., preferably at least 400° C.
   56. The process according to any one of the previous statements, wherein the temperature during step f) is at most 1600° C., preferably at most 1400° C., preferably at most 1200° C., preferably at most 1000° C., preferably at most 800° C., preferably at most 600° C.
   57. The process according to any one of the previous statements, wherein the temperature during step f) is at least 150° C. to at most 1600° C., preferably at least 200° C. to at most 1400° C., preferably at least 250° C. to at most 1200° C., preferably at least 300° C. to at most 1000° C., preferably at least 350° C. to at most 800° C., preferably at least 400° C. to at most 600° C.
   58. The process according to any one of the previous statements, wherein the pressure during step f) is at least 0.5 bar, preferably at least 0.8 bar, preferably at least 0.9 bar, preferably at least 1.0 bar, preferably at least 1.1 bar, preferably at least 5.0 bar, preferably at least 10.0 bar.
   59. The process according to any one of the previous statements, wherein the pressure during step f) is at most 50.0 bar, preferably at most 45.0 bar, preferably at most 40.0 bar, preferably at most 35.0 bar, preferably at most 30.0 bar, preferably at most 25.0 bar, preferably at most 20.0 bar, preferably at most 15.0 bar.
   60. The process according to any one of the previous statements, wherein the pressure during step f) is at least 0.5 bar to at most 50.0 bar, preferably at least 0.8 bar to at most 45.0 bar, preferably at least 0.9 bar to at most 40.0 bar, preferably at least 1.0 bar to at most 35.0 bar, preferably at least 1.1 bar to at most 30.0 bar, preferably at least 5.0 bar to at most 25.0 bar, preferably at least 10.0 bar to at most 20.0 bar, preferably at least 10.0 bar to at most 15.0 bar.
   61. The process according to any one of the previous statements, further comprising the step of returning the halogen reactant (X₂) obtained in step e) and/or step f), to step b) of the process.
   62. The process according to any one of the previous statements, wherein halogen reactant (X₂) obtained in step e) and/or step f) is purified before being fed into step b), and preferably purified by condensation of the halogen reactant (X₂).
   63. The process according to any one of the previous statements, wherein the halogen reactant (X₂) obtained in step e) and/or step f), is dried before being fed into step b), preferably dried over molecular sieves.
   64. The process according to any one of the previous statements, wherein step e) of said process comprises supplying an aqueous solution of hydrogen halide (HX) to an electrolysis cell containing positive and negative electrodes, and decomposing said hydrogen halide electrolytically by maintaining an electrical potential from about 0.5 to 2.5 V between said electrodes.
   65. The process according to any one of the previous statements, wherein step e) of said process comprises supplying an aqueous solution of hydrogen halide (HX) to an electrolysis cell containing positive and negative electrodes, and decomposing said hydrogen halide electrolytically by maintaining a current density from about 100 to 800 mA/cm² between said electrodes.
   66. The process according to any one of the previous statements, wherein said electrolysis cell is a polymer electrolyte membrane cell (PEM) containing at least a proton-conductive membrane.
   67. The process according to any one of the previous statements, wherein said hydrogen halide is decomposed in step e) at a temperature of from 20 to 95° C.
   68. The process according to any one of the previous statements, wherein said hydrogen halide is decomposed in step e) at a pressure of from 1 to 50 bar.
   69. The process according to any one of the previous statements, wherein the gaseous hydrogen (H₂) stream generated in step e) comprises at least 90.0 mol %, such as at least 95.0 mol %, or at least 99.0 mol %, or at least 99.5 mol %, or at least 99.9 mol % hydrogen.
   70. A process for producing dimethyl ether (DME) and hydrogen (H₂) from methane, according to any one of the previous statements comprising the steps of:
a) providing a gaseous feed stream comprising methane,
b) reacting said gaseous feed stream with bromide (Br₂) under reaction conditions effective to produce an effluent stream comprising methyl bromide (MeBr) and hydrogen bromide (HBr), and wherein said effluent stream optionally comprises unreacted methane and optionally polybrominated alkanes;
c) separating from the effluent stream obtained in b):
   (i) a methyl bromide (MeBr) stream, optionally comprising unreacted methane; and,
   (ii) a hydrogen bromide (HBr) stream;
   (iii) optionally, a polybrominated alkanes stream;
d) reacting the methyl bromide (MeBr) stream separated in step c) with a solid metal oxide (MO₍ₛ₎), under reaction conditions effective to produce metal bromide (MBr) and dimethyl ether (DME); and,
e) decomposing by means of electrolysis said hydrogen bromide (HBr) stream separated in step c) under conditions effective to produce a gaseous hydrogen (H₂) stream and a stream comprising bromine (Br₂), and
f) Optionally, regenerating the metal bromide (MBr) to metal oxide (MO) and bromine (Br₂), preferably by contacting the metal bromide (MBr) with oxygen (O₂) or air, thereby forming metal oxide (MO) and bromine (Br₂), and
g) Optionally returning the bromine (Br₂) obtained in step e) and/or step f), to step b) of the process, and
h) Optionally, recovering a stream comprising dimethyl ether and optionally unreacted methane after step d).
   71. The process according to any one of the previous statements, wherein the process further comprises a step of removing, preferably distilling, unreacted methane from the effluent stream.
   72. The process according to any one of the previous statements, wherein the process further comprises a step of removing, preferably distilling, unreacted methane from the methyl halide (MeX) stream.
   73. The process according to any one of the previous statements, wherein the process further comprises a step of removing, preferably distilling, unreacted methane, from the dimethyl ether produced in step d).
   74. The process according to any one of the previous statements, wherein removed unreacted methane is recycled or fed into step a) and/or step b) of the process.
   75. The process according to any one of the previous statements, wherein the process is a continuous process.
   76. System for producing dimethyl ether (DME) and hydrogen (H₂) from methane, comprising;
a halogenation reactor (1), configured to react a gaseous feed stream comprising methane with at least one halogen reactant (X₂) into an effluent stream comprising methyl halide (MeX), and hydrogen halide (HX), and optionally unreacted methane,
a separation system (2), which is fluidly connected to said halogenation reactor (1) and configured to receive an effluent stream from said halogenation reactor (1) and configured to separate a hydrogen halide (HX) stream and a methyl halide (MeX) stream from said effluent stream;
a dimethyl ether synthesis reactor (3), which is fluidly connected to said separation system (2), and configured to receive a methyl halide (MeX) stream from said separation system (2) and to react said methyl halide (MeX) stream with a solid metal oxide (MO₍ₛ₎) into metal halide (MX) and dimethyl ether (DME); and
an electrolysis unit (4) comprising at least one electrolysis cell and a power source for supplying current to said electrolysis cell, which is fluidly connected to said separation system (2), and configured to receive a hydrogen halide (HX) stream and to decompose said hydrogen halide (HX) stream into a gaseous hydrogen (H₂) stream and a stream comprising halogen reactant (X₂);
a feed stream supply system (5) for supplying a gaseous feed stream comprising methane to said halogenation reactor (1);
a halogen supply system (6) for supplying a halogen reactant (X₂) to said halogenation reactor (1);
an effluent recovery system (10), configured to recover an effluent stream from said halogenation reactor (1), and for feeding said recovered effluent stream to said separation system (2);
a dimethyl ether recovery system (7), configured to recover a stream comprising dimethyl ether (DME) from said dimethyl ether synthesis reactor (3);
a hydrogen recovery system (8), configured to recover a gaseous hydrogen (H₂) stream from said electrolysis unit (4);
a halogen recovery system (9), configured to recover halogen reactant (X₂) from said electrolysis unit (4);
a hydrogen halide transfer system (12), configured to recover a hydrogen halide (HX) stream from said separation system (2), and to supply said recovered hydrogen halide (HX) stream to the electrolysis unit (4);
a methyl halide transfer system (11), configured to recover a methyl halide (MeX) stream from said separation system (2), and to supply said recovered methyl halide (MeX) stream to the dimethyl ether synthesis reactor (3),
optionally, an absorbent supply line (17), for supplying an aqueous absorbent, preferably an aqueous hydrogen halide (HX) solution, to said separation system (2);
optionally, a polyhalogenated alkanes removal unit, configured to receive effluent stream from said halogenation reactor (1) and to remove polyhalogenated alkanes from said effluent stream, and,
optionally, a C₂₊ alkyl monohalide removal unit, configured to receive effluent stream from said halogenation reactor (1) and to remove C₂₊ alkyl monohalides from said effluent stream.

In a first aspect, the invention provides a process for producing dimethyl ether (DME) and hydrogen (H₂) from methane. The process in particular comprises the steps of:
a) providing a gaseous feed stream comprising methane;
b) reacting said gaseous feed stream with at least one halogen reactant (X₂), under reaction conditions effective to produce an effluent stream comprising methyl halide (MeX), and hydrogen halide (HX), and wherein said effluent stream optionally comprises unreacted methane;
c) separating from the effluent stream obtained in step b):
   (i) a methyl halide (MeX) stream, optionally comprising unreacted methane; and,
   (ii) a hydrogen halide (HX) stream;
d) reacting the methyl halide (MeX) stream separated in step c) with a solid metal oxide (MO₍ₛ₎), under reaction conditions effective to produce metal halide (MX) and dimethyl ether (DME); and,
e) decomposing by means of electrolysis said hydrogen halide (HX) stream separated in step c) under conditions effective to produce a gaseous hydrogen (H₂) stream and a stream comprising halogen reactant (X₂).

In particular, the present invention thereto provides a process for producing dimethyl ether (DME) and hydrogen (H₂) from methane, comprising the steps of:
a) providing a gaseous feed stream comprising methane;
b) reacting said gaseous feed stream with at least one halogen reactant (X₂), under reaction conditions effective to produce an effluent stream comprising methyl halide (MeX), and hydrogen halide (HX), optionally polyhalogenated alkanes, and optionally unreacted methane;
c) separating from the effluent stream obtained in step b):
   (i) a methyl halide (MeX) stream, optionally comprising unreacted methane; and,
   (ii) an aqueous hydrogen halide (HX) stream or a hydrogen halide (HX) stream; d) reacting the methyl halide (MeX) stream separated in step c) with a solid metal oxide (MO₍ₛ₎), under reaction conditions effective to produce metal halide (MX) and dimethyl ether (DME); and,
e) decomposing by means of electrolysis said aqueous hydrogen halide (HX) stream, or said hydrogen halide (HX) stream separated in step c) under conditions effective to produce a gaseous hydrogen (H₂) stream and a stream comprising halogen reactant (X₂);
wherein in step c) the hydrogen halide (HX) stream is separated from the effluent stream by contacting the effluent stream comprising said hydrogen halide with an aqueous absorbent, or by distillation, thereby forming respectively an aqueous hydrogen halide (HX) stream or a hydrogen halide (HX) stream.

More specifically, a process for producing dimethyl ether (DME) and hydrogen (H₂) from methane according to the invention, comprising the steps of:
a) providing a gaseous feed stream comprising methane;
b) reacting said gaseous feed stream with at least one halogen reactant (X₂), under reaction conditions effective to produce an effluent stream comprising methyl halide (MeX), hydrogen halide (HX), optionally polyhalogenated alkanes, and optionally unreacted methane;
c) separating from the effluent stream obtained in step b):
   (i) a methyl halide (MeX) stream, optionally comprising unreacted methane; and,
   (ii) a hydrogen halide (HX) stream;
   (iii) optionally, a polyhalogenated alkanes stream:
d) reacting the methyl halide (MeX) stream separated in step c) with a solid metal oxide (MO₍ₛ₎), under reaction conditions effective to produce metal halide (MX) and dimethyl ether (DME);
e) decomposing by means of electrolysis said hydrogen halide (HX) stream separated in step c) under conditions effective to produce a gaseous hydrogen (H₂) stream and a stream comprising halogen reactant (X₂);
f) regenerating the metal halide (MX) to metal oxide (MO) and halogen reactant (X₂), preferably by contacting the metal halide (MX) with oxygen (O₂) or air, thereby forming metal oxide (MO) and halogen reactant (X₂); and
g) optionally returning the halogen reactant (X₂) obtained in step e) and/or step f), to step b) of the process.

It is preferred according to the invention that steps b) and d) are performed in separate reaction steps, preferably in separate reactors or separate reaction zones. The process of the invention is preferably a continuous process.

The present invention provides a process for producing streams of dimethyl ether and hydrogen, starting from a gaseous feed stream that comprises or essentially consists of methane, such as a biogas or a natural gas. In other words, the present invention provides a process for producing dimethyl ether from a gaseous stream comprising methane which is not syngas.

The present process comprises the reaction of the gaseous feed stream with at least one halogen reactant to form methyl halide and hydrogen halide, followed by a reaction of the produced methyl halide in the presence of a solid metal oxide into a metal halide and dimethyl ether. The present process is further characterised in that it comprises the step of separating the methyl halide, produced by reacting the gaseous feed stream with a halogen reactant, from other halogen-containing compounds, and in particular from the hydrogen halide produced in this step, before subjecting the produced methyl halide to an ether synthesis reaction in the presence of said solid metal oxide. Such separation step upstream of the ether synthesis step advantageously and unexpectedly allows to efficiently convert the methyl halide stream to a dimethyl ether stream, with minimal to no production of undesired side-products, such as e.g. methanol (MeOH).

In addition, the present process provides a step wherein the separated hydrogen halide may be treated electrolytically to be decomposed in separate streams of hydrogen and halogen reactant, yielding a valuable side product, i.e. hydrogen. Moreover, the recovered halogen reactant may advantageously be re-used in the present process.

The present process thus provides an efficient process for the conversion of methane in dimethyl ether, with the production hydrogen which is an industrially relevant side product.

A first step of the process comprises the provision of a gaseous feed stream comprising methane.

The term “gaseous feed stream” and “reaction gas” are used herein as synonyms and refers to a gaseous stream comprising methane.

In certain embodiments of a process according to the invention, the applied gaseous feed stream may also comprise C₂₊ alkanes.

The term “alkane” refers to an organic compound being either an acyclic saturated hydrocarbon or a cyclic saturated hydrocarbon. As used herein alkanes might be straight, branched or cyclic (but not aromatic). Preferably, the alkanes are straight or branched. In preferred embodiments, the alkanes are acyclic saturated hydrocarbon and fulfil the following formula CᵢH₍₂ᵢ₊₂₎,wherein i is an integer. Examples are methane, ethane, propane, butane, pentane, and hexane.

The term “C₂₊ alkanes” as used herein intends to refer to alkanes having more than two carbon atoms. In some embodiments, the C₂₊ alkanes are C₂₋₆ alkanes, i.e. alkanes comprising two to six carbon atoms, thus for instance C₂, C₃, C₄, C₅, C₆ alkanes, and any mixtures thereof. When a subscript is used herein following a carbon atom, the subscript refers to the number of carbon atoms that the named group may contain. Examples of C₂₊ alkanes are ethane, propane, butanes, pentanes, the hexanes, and mixtures of two or more of these.

For instance, in certain embodiments the applied gaseous feed stream may comprises a mixture of methane and ethane, or a mixture of methane and propane, or a mixture of methane, ethane and propane.

Preferably, a gaseous feed stream comprising methane for use in the present process, comprises at least 75.0 vol % methane, preferably at least 80.0 vol % methane, preferably at least 85.0 vol % methane, preferably at least 90.0 vol % methane, preferably at least 95.0 vol % methane, preferably at least 98.0 vol % methane, preferably at least 99.0 vol % methane, preferably at least 99.9 vol % methane, based on the total volume of the gaseous feed stream.

Optionally, a gaseous feed stream as applied in a process according to the invention may also comprise hydrogen. Preferably, the molar ratio of this optional hydrogen to methane in said gaseous feed stream may be in the range from about 1:4 to 0:1.

Optionally, a gaseous feed stream as applied in a process according to the invention may also comprise minor amounts of other components selected from oxygen, nitrogen, or carbon dioxide. Preferably such other component may be present in an amount lower than 2.0 mol %, such as lower than 1.5 mol %, or lower than 1.0 mol % or lower than 0.5 mol %, based on the total gaseous feed stream. In certain embodiments, the composition of the gaseous feed stream is comparable to the composition of a natural gas or a biogas, or a mixture thereof.

In certain embodiments, the gaseous feed stream applied in step a) of the process comprises a natural gas, a biogas; a refinery gas (i.e. the gas fraction coming from the refinery of crude oil) or any mixtures thereof. Preferably, the gaseous feed stream comprises natural gas and/or biogas.

In certain embodiments of a process according to the invention, the gaseous feed stream applied in step a) of the process gas is a natural gas. The term “natural gas” refers to a multi-component gas obtained from a crude oil well (associated gas) or from a subterranean gas-bearing formation (non-associated gas). The composition and pressure of natural gas can vary significantly. A typical natural gas stream contains methane as a significant component.

Natural gas may also contain ethane, higher molecular weight hydrocarbons, such as C2+ alkanes, acid gases (such as carbon dioxide, hydrogen sulphide, carbonyl sulphide, carbon disulphide, and mercaptans), and minor amounts of contaminants such as water, nitrogen, iron sulphide, wax, and crude oil. As used herein, “natural gas” may also include gas resulting from the regasification of a liquefied natural gas, which has been purified to remove contaminates, such as water, acid gases, and most of the higher molecular weight hydrocarbons (e.g. C₁₂+ hydrocarbons). Conventional methods can be used for removing impurities and/or adjusting the relative amount of hydrocarbon compounds present in the gaseous feed stream.

In certain embodiments of a process according to the invention, the gaseous feed stream applied in step a) of the process gas is a biogas or a biomethane produced from biogas. The term “biogas” refers to a multi-component gas, primarily consisting of methane and carbon dioxide, produced from anaerobic digestion or by methanogenesis from raw materials such as but not limited to agricultural waste, manure, municipal waste, plant material, sewage, green waste or food waste. Biogas may be purified to remove carbon dioxide, oxygen, sulphur and/or silicon containing compounds to produce biomethane, which could be applied as gaseous feed stream in the present process.

In certain embodiments, said natural gas may be of fossil origin. In certain embodiments, said natural gas may be of renewable origin. Natural gas of renewable origin for instance includes gas produced from existing waste streams and a variety of renewable and sustainable biomass sources, including but not limited to animal waste, crop residuals and food waste, organic waste from dairies and farms, and naturally-occurring biological breakdown of organic waste at facilities such as wastewater treatment plants and landfills. In certain embodiments, said natural gas may comprise a combination of natural gas from fossil origin and from renewable source.

Preferably, the gaseous feed stream applied in step a) of the process is substantially free of sulphur species, e.g. it contains less than 1.0 mol %, or less than 0.5 mol %, or less than 0.1 mol %, or less than 0.01 mol %, or less than 0.001 mol %, or less than 0.0001 mol %, or less than 0.00001 mol % of sulphur species. In an example, the gaseous feed stream is free of sulphur species. Sulphur species may for instance refer to species in the form of H₂S, COS, CS₂. Desulphurisation, if necessary, can be achieved by adsorption and/or absorption.

In certain other embodiments of a process according to the invention, the gaseous feed stream consists of methane.

Step b) of a process of the invention comprises reacting the gaseous feed stream as defined herein with at least one halogen reactant (X₂), under reaction conditions effective to produce an effluent stream comprising methyl halide (MeX), and hydrogen halide (HX). The effluent stream may optionally comprise unreacted methane.

The halogen reactant applied in this step of the present process may be selected from the group consisting of bromine (Br₂), chlorine (Cl₂), fluorine (F₂), and iodine (I₂), and preferably is bromine. Especially, the recovery of bromine in the process e) and f) requires less energy, reagents and/or equipment.

Therefore, in a preferred embodiment, the invention provides a process for producing dimethyl ether (DME) and hydrogen (H₂) from methane, comprising the steps of:
a) providing a gaseous feed stream comprising methane;
b) reacting said gaseous feed stream with bromine (Br₂), under reaction conditions effective to produce an effluent stream comprising methyl bromide (MeBr), hydrogen bromide (HBr), and wherein the effluent stream optionally comprises unreacted methane;
c) separating the hydrogen bromide (HBr) from said effluent stream, thereby recovering a methyl bromide (MeBr) stream;
d) reacting the methyl bromide (MeBr) stream separated in step c) with a solid metal oxide (MO₍ₛ₎), under reaction conditions effective to produce metal bromide (MBr) and dimethyl ether (DME);
e) decomposing by means of electrolysis said hydrogen bromide (HBr) separated in step c) under conditions effective to produce a gaseous hydrogen (H₂) stream and a stream comprising bromine (Br₂);
f) optionally, regenerating the metal bromide (MBr) to metal oxide (MO) and bromine (Br₂); and,
g) optionally returning the bromine (Br₂) obtained in step e) and/or step f), to step b) of the process.

Preferably, the halogen reactant, such as bromine, is introduced directly into the reaction zone. The halogen reactant may be introduced in the reaction zone in liquid state. The halogen reactant may be introduced in the reaction zone in pure form, or dissolved in an aqueous hydrogen halide solution.

In accordance with the present process, step b) of the process is preferably carried at a temperature during step b) of at least 150° C., preferably at least 200° C., preferably at least 250° C., preferably at least 300° C., preferably at least 350° C., preferably at least 380° C. In accordance with the present process, step b) of the process is preferably carried at a temperature during step b) of at most 900° C., preferably at most 800° C., preferably at most 700° C., preferably at most 600° C., preferably at most 550° C., preferably at most 520° C.

For instance, preferred temperature conditions for the reaction between the feed stream comprising methane and the halogen reactant in the present process are comprised between 150° C. and 900° C., preferably comprised between 200° C. and 800° C., preferably comprised between 250° C. and 700° C., preferably comprised between 300° C. and 600° C., preferably comprised between 350° C. and 550° C., preferably comprised between 380° C. and 520° C. In some embodiments, the exothermic nature of the reaction is sufficient to maintain the temperature during the reaction.

In accordance with the present process, step b) of the process is carried out at a reaction pressure of at least 1.0 bar, preferably at least 1.5 bar, preferably at least 2.0 bar, preferably at least 2.5 bar, preferably at least 3.0 bar, preferably at least 5.0 bar, preferably at least 10.0 bar. In accordance with the present process, step b) of the process is carried out at a reaction pressure of at most 50.0 bar, preferably at most 45.0 bar, preferably at most 40.0 bar, preferably at most 35.0 bar, preferably at most 30.0 bar, preferably at most 25.0 bar, preferably at most 20.0 bar.

For instance, preferred pressure conditions for the reaction between the feed stream comprising methane and the halogen reactant in the present process are comprised between 1.0 bar and 50.0 bar, preferably comprised between 1.5 bar and 45.0 bar, preferably comprised between 2.0 bar and 40.0 bar, preferably comprised between 2.5 bar and 35.0 bar, preferably comprised between 3.0 bar and 30.0 bar, preferably comprised between 5.0 bar and 25.0 bar, preferably comprised between 10.0 bar and 20.0 bar.

In certain embodiments of the present process, the feed stream comprising methane and the halogen reactant are applied at a molar ratio of methane/halogen comprised between 2/1 and 7/1, preferably between 3/1 to 6/1, preferably between 4/1 to 5/1.

The time required for the gaseous feed stream and halogen to react will vary depending on the specific feed material utilized, the halogen utilized, and the temperature of the reaction. In an example, the gaseous feed stream is contacted in step b) with the halogen reactant (X₂), at a temperature of between 380° C. and 520° C., at a pressure of between 10 bar and 20 bar, and for at least 0.1 min to at most 2.0 min.

The effluent stream resulting from reaction step b) mainly comprises methyl halide and hydrogen halide. This effluent stream may also comprise certain amounts of unreacted methane. Also, depending on the composition of the initial gaseous feed stream applied in the process, the effluent stream may comprise certain amounts of C₂₊ alkyl monohalides and/or certain amounts of polyhalogenated alkanes such as polyhalogenated methane and/or polyhalogenated C₂₊ alkanes, that have been formed during reaction step b).

In a next step, the process of the invention comprises the step of separating from the effluent stream obtained in step b): a methyl halide (MeX) stream and a hydrogen halide (HX) stream. The methyl halide stream is then further reacted with a solid metal oxide (in step d) of the process), while the hydrogen halide stream is electrolysed (in step e) of the process).

In preferred embodiments, a hydrogen halide (HX) stream is separated from the effluent stream in step c) of the present process, by contacting the effluent stream comprising said hydrogen halide with an aqueous absorbent, capable of absorbing said hydrogen halide (HX). For instance, the hydrogen halide (HX) stream may be separated from the effluent stream in a separation system, e.g. in a quench column, preferably by contacting the gaseous effluent stream with a suitable aqueous absorbent, preferably counter-currently.

Preferably, the aqueous absorbent applied in step c) of the present process is an aqueous hydrogen halide (HX) solution.

In certain preferred embodiments of the present invention, the aqueous absorbent applied in the present process is an aqueous hydrogen halide (HX) solution, wherein the halide anion applied in said solution, e.g. a bromide anion, and the halogen reactant applied in step b) of the process are the same halogen, for instance bromine. Hence, in certain preferred embodiments, the halogen reactant applied in step a) is bromide (Br2) and the hydrogen halide solution applied in step c) is a hydrogen bromide (HBr) solution.

Preferably, a hydrogen halide solution as applied during separation step c) contain a hydrogen halide (HX) weight fraction of at least 10.0% by weight to at most 40.0% by weight, such as of at least 15.0% by weight to at most 35.0% by weight, or of at least 20.0% by weight to at most 30.0% by weight, or of at least 25.0% by weight to at most 30.0% by weight, wherein said weight % is expressed based on total hydrogen halide solution.

In certain embodiments of a process of the invention, depending on the composition of the initial gaseous feed stream applied in the process, the effluent stream may comprise additional components besides the methyl halide, the hydrogen halide, and optional unreacted methane.

In certain embodiments of a process of the invention, polyhalogenated alkanes are formed in step b) and are present in the effluent stream obtained after step b).

The term “polyhalogenated alkanes” as used herein refers to alkanes wherein more than one hydrogen atom is replaced by a halogen atom (X), wherein X is preferably selected from the group consisting of Br, Cl, F, I and At, more preferably X is Br. “Polyhalogenated alkanes” as used herein preferably refer to polyhalogenated alkanes such as polyhalogenated C₁-C₆ alkanes, preferably polyhalogenated C₁-C₄ alkanes, preferably polyhalogenated C₁-C₃ alkanes. When a subscript is used herein following a carbon atom, the subscript refers to the number of carbon atoms that the named group may contain.

In certain embodiments, the term “polyhalogenated alkane” refers to a compound with the following formula (1):

CₙH₍₍₂ₙ₊₂₎₋ₘ₎Xₘ (I)
wherein:
   n is an integer and n≥1, preferably 1≤n≥6; preferably 1≤n≥4; preferably 1≤n≥3;
   m is an integer and m≥2; preferably 2≤m≥4, preferably 2≤m≥3; and,
   X is selected from the group consisting of Br, Cl, F, I and At, preferably X is Br.

In certain embodiments, polyhalogenated alkanes comprise polyhalogenated C₁ alkanes.

In certain embodiments, polyhalogenated alkanes comprise polyhalogenated C₂₊ alkanes.

In certain embodiments, polyhalogenated alkanes comprise polyhalogenated C₁ alkanes and polyhalogenated C₂₊ alkanes.

The terms “polyhalogenated C₁ alkane” and “polyhalogenated methane” are used herein interchangeably and intend to refer to a C₁ alkane wherein more than one hydrogen atom is replaced by a halogen atom (X), wherein X is preferably selected from the group consisting of Br, Cl, F, I and At, more preferably wherein X is Br.

In certain embodiments, the terms “polyhalogenated C₁ alkane” and “polyhalogenated methane” refer to a compound with the following formula (II):

C₁H₍₄₋ₚ₎Xₚ (II)
wherein:
   p is an integer and p≥2; preferably 2≤p≥4, preferably 2≤p≥3; and,
   X is selected from the group consisting of Br, Cl, F, I and At, and preferably X is Br.

In certain preferred embodiments, a “polyhalogenated methane” is selected from the group comprising CH₂X₂, CHX₂, and CX₄, and any mixtures thereof, wherein X is as defined herein above. For instance, polyhalogenated methanes formed in step b) of the process comprise methylene dihalides (CH₂X₂). Preferred examples of polyhalogenated methanes include but are not limited to CH₂Br₂, CHBr₃, and CBr₄.

The term “polyhalogenated C₂₊ alkanes” as used herein refers to C₂₊ alkanes wherein more than one hydrogen atom is replaced by a halogen atom (X), wherein X is preferably selected from the group consisting of Br, Cl, F, I and At, more preferably wherein X is Br. Preferably, polyhalogenated C₂₊ alkanes preferably refer to polyhalogenated C₂₊ alkanes such as polyhalogenated C₂-C₆ alkanes, preferably polyhalogenated C₂-C₄ alkanes, preferably polyhalogenated C₂-C₃ alkanes.

In certain embodiments, a “polyhalogenated C₂₊ alkane” refers to a compound with the following formula (III):

C_{q}H_{((2q+2)-r)}Xᵣ (III)
wherein:
   q is an integer and q≥2, preferably 2≤q≥6; preferably 2≤q≥4; preferably 2≤q≥3;
   r is an integer and r≥2; preferably 2≤r≥4, preferably 2≤r≥3; and,
   X is selected from the group consisting of Br, Cl, F, I and At, preferably X is Br.

For instance, polyhalogenated C₂₊ alkanes formed in step b) of the process may include a polyhalogenated C₂ alkane (such as e.g. dibromoethane) and/or a polyhalogenated C₃ alkane (such as e.g. dibromopropane).

In certain embodiments of a process of the invention, C₂₊ alkyl monohalides are also formed in step b) and are present in the effluent stream obtained after step b).

The terms “C₂₊ alkyl monohalides” and “monohalogenated C₂₊ alkanes” are used herein interchangeably and refer to C₂₊ alkanes wherein a single hydrogen atom is replaced by a halogen atom (X), wherein X is preferably selected from the group consisting of Br, Cl, F, I and At, more preferably wherein X is Br. Preferably, the monohalogenated C₂₊ alkanes preferably refer to monohalogenated C₂₊ such as monohalogenated C₂-C₆ alkanes, preferably monohalogenated C₂-C₄ alkanes, preferably monohalogenated C₂-C₃ alkanes.

In other words, the terms “C₂₊ alkyl monohalide” and “monohalogenated C₂₊ alkane” refer to a compound with the following formula (IV):

CₛH₍₂ₛ₊₁₎X₁ (IV)
wherein:
   s is an integer and s≥2; preferably 2≤s≥6; preferably 2≤s≥4; preferably 2≤s≥3 and,
   X is selected from the group consisting of Br, Cl, F, I and At, preferably X is Br.

For instance, C₂₊ alkyl monohalides formed in step b) of the process may include a C₂ alkyl monohalide (e.g. ethyl bromide) and/or a C₃ alkyl monohalide (such as e.g. n-propyl bromide).

In view thereof, the present process may comprise a separation of said polyhalogenated alkanes and/or C₂₊ alkyl monohalides from said effluent gas. Therefore, in certain embodiments, step c) in a process of the invention comprises separating from the effluent stream obtained in step b):
a methyl halide (MeX) stream; optionally comprising unreacted methane and/or optionally C₂₊ alkyl monohalides;
a hydrogen halide (HX) stream; and,
a polyhalogenated alkanes stream.

In some embodiments, the C₂₊ alkyl monohalides may be separated from the effluent stream in a separate stream, i.e. in a C₂₊ alkyl monohalides stream.

In some embodiments, a polyhalogenated alkanes stream is separated from the effluent stream, preferably by distillation, as:
one stream, which may be further separated into a polyhalogenated methane stream and a polyhalogenated C₂₊ alkanes stream, preferably by distillation; or,
a polyhalogenated methane stream and a polyhalogenated C₂₊ alkanes stream.

In some embodiments, step c) comprises a single separation step of the effluent stream, i.e. the effluent stream is separated in one step. In some embodiments, step c) comprises two separation steps, i.e. a hydrogen halide (HX) separation step, and a polyhalogenated alkanes separation step. In some embodiments, step c) comprises three separation steps, i.e. a hydrogen halide (HX) separation step, a polyhalogenated alkanes separation step, and a C₂₊ alkyl monohalide separation step.

In one example, step c) in a process of the invention comprises separating from the effluent stream in obtained in step b):
a methyl halide (MeX) stream; optionally comprising unreacted methane and/or optionally comprising C₂₊ alkyl monohalides;
a hydrogen halide (HX) stream; and,
a polyhalogenated alkanes stream.

In another example, step c) in a process of the invention comprises separating from the effluent stream in obtained in step b):
a methyl halide (MeX) stream; optionally comprising unreacted methane and/or optionally comprising C₂₊ alkyl monohalides;
a hydrogen halide (HX) stream; and,
a polyhalogenated methane stream; and,
a polyhalogenated C₂₊ alkanes stream.

In another example, step c) in a process of the invention comprises separating from the effluent stream in obtained in step b):
a methyl halide (MeX) stream; optionally comprising unreacted methane;
a hydrogen halide (HX) stream; and,
a C₂₊ alkyl monohalides stream.

In another example, step c) in a process of the invention comprises separating from the effluent stream in obtained in step b):
a methyl halide (MeX) stream; optionally comprising unreacted methane;
a hydrogen halide (HX) stream;
a polyhalogenated alkanes stream; and,
a C₂₊ alkyl monohalides stream.

In another example, step c) in a process of the invention comprises separating from the effluent stream in obtained in step b):
a methyl halide (MeX) stream; optionally comprising unreacted methane;
a hydrogen halide (HX) stream;
a polyhalogenated methane stream;
a polyhalogenated C₂₊ alkanes stream; and,
a C₂₊ alkyl monohalides stream.

Separation of polyhalogenated alkanes from the effluent stream has the benefit of allowing to recover a purified methyl halide (MeX) stream, which contains less or no other polyhalogenated alkanes such as polyhalogenated methane and/or polyhalogenated C₂+ alkanes, and hence such purified methyl halide (MeX) stream can advantageously be converted into a stream of methanol, without the generation of significant amounts of non-alcohol products apart from alkanes. This way, a methanol can be produced with less than 10.000 ppm, preferably less than 7.000 ppm, preferably less than 5.000 ppm, preferably less than 3.000 ppm, preferably less than 2.000 ppm, preferably less than 1.000 ppm non-alcoholic products apart from alkanes.

In particular, a preferred process for producing methanol (MeOH) and hydrogen (H₂) from methane according to the invention, comprises the steps of:
a) providing a gaseous feed stream comprising methane, and optionally comprising C₂+ alkanes;
b) reacting said gaseous feed stream with at least one halogen reactant (X₂), under reaction conditions effective to produce an effluent stream comprising methyl halide (MeX), hydrogen halide (HX), and wherein said effluent stream optionally comprises unreacted methane; C₂₊ alkyl monohalides and/or polyhalogenated alkanes, such as polyhalogenated methane and/or polyhalogenated C₂₊ alkanes;
c) separating from the effluent stream obtained in step b):
   (i) a methyl halide (MeX) stream, optionally comprising unreacted methane;
   (ii) a hydrogen halide (HX) stream;
   (iii) optionally a polyhalogenated alkanes stream, which may comprise a polyhalogenated methane stream and/or polyhalogenated C₂₊ alkanes stream; and,
   (iv) optionally a C₂₊ alkyl monohalides stream.
d) reacting the methyl halide (MeX) stream separated in step c) with a solid metal oxide (MO₍ₛ₎), under reaction conditions effective to produce metal halide (MX) and dimethyl ether (DME);
e) decomposing by means of electrolysis said hydrogen halide (HX) stream separated in step c) under conditions effective to produce a gaseous hydrogen (H₂) stream and a stream comprising halogen reactant (X₂);
f) optionally regenerating the metal halide (MX) to metal oxide (MO) and halogen reactant (X₂);
g) optionally returning the halogen reactant (X₂) obtained in step e) and or step f), to step b) of the process; and,
h) optionally, converting the polyhalogenated alkanes stream and/or the C₂₊ alkyl monohalides stream into their corresponding alcohols.

Preferably, polyhalogenated alkanes formed in step b) of the process are separated from the effluent stream by distillation. For instance, a polyhalogenated alkanes stream or a polyhalogenated methane stream and a polyhalogenated C₂₊ alkanes stream can be separated from the effluent stream in a separation column. Preferably, as there is a large difference in molecular weight between the polyhalogenated alkanes and the mono-halogenated compounds, and thus a large difference in boiling point, distillation may be a preferred separation technique. Preferably polyhalogenated alkanes are separated from the effluent stream before the mono-halogenated compounds, i.e. methyl halide, hydrogen halide and C₂₊ alkyl monohalides, are separated from the effluent stream.

In certain preferred embodiments of the present invention, the process comprises the step of separating polyhalogenated alkanes formed in step b) from said effluent stream, thereby creating a polyhalogenated alkanes stream, whereby said polyhalogenated alkanes formed in step b) are separated from said effluent stream prior to separating hydrogen halide from said effluent stream.

Preferably, C₂₊ alkyl monohalides formed in step b) of the process are separated from the effluent stream by distillation. For instance, a C₂₊ alkyl monohalide stream can be separated from the effluent stream in a separation column

In certain preferred embodiments of the present invention, the process comprises the step of separating C₂₊ alkyl monohalides formed in step b) of the process from said effluent stream, thereby creating a C₂₊ alkyl monohalide stream, whereby said C₂₊ alkyl monohalides formed in step b) are separated from said effluent stream prior to separating hydrogen halide from said effluent stream, preferably by distillation.

In certain embodiments, the present process comprises the separation of polyhalogenated methane stream and polyhalogenated C₂₊ alkanes stream in separate steps and/or in separate separation columns. Alternatively, the present process may comprise the separation of polyhalogenated methane stream and polyhalogenated C₂₊ alkanes stream in a single step and/or in single separation column.

In certain embodiments, the present process comprises the separation of polyhalogenated alkanes stream and methyl halide stream in separate steps and/or in separate separation columns. Alternatively, the present process may comprise the separation of polyhalogenated alkanes stream and methyl halide stream in a single step and/or in a single separation column.

The methyl halide (MeX) stream separated in step c) is preferably a gaseous stream.

Upon separating the hydrogen halide (HX), and optionally polyhalogenated alkanes stream and/or C₂₊ alkyl monohalide streams, from said effluent stream in step c), the present process allows to recover a stream which mainly comprises methyl halide (MeX). Preferably the separated methyl halide stream comprises at least 75.0 mol %, such as least 80.0 mol %, or at least 85.0 mol %, or at least 90.0 mol % of methyl halide, based of the total amount of said stream. It will be noted however that the separated methyl halide stream may still comprise certain amounts of unreacted methane.

Preferably, the methyl halide (MeX) stream separated in step c) is essentially free of hydrogen halide. In this context, the term “essentially free of hydrogen halide” intends to indicate that, based on the total amount of the stream, the amount of hydrogen halide in said stream is less than 10.00 mol %, such as less than 8.00 mol %, or less than 5.00 mol %, or less than 3.00 mol %, or less than 1.00 mol %, or less than 0.10 mol %, preferably less than 0.05 mol %, preferably less than 0.01 mol %.

In accordance with the present invention, the methyl halide (MeX) stream separated in step c), is also preferably “essentially free” of polyhalogenated alkanes. “Essentially free of polyhalogenated alkanes” in this context means that the methyl halide (MeX) stream obtained in step c), comprises less than 1.0 mol %, such as less than 0.1 mol %, preferably less than 0.5 mol % polyhalogenated alkanes, as defined herein.

In accordance with the present invention, the methyl halide (MeX) stream separated in step c), is also preferably “essentially free” of polyhalogenated methane. “Essentially free of polyhalogenated methane” in this context means that the methyl halide (MeX) stream obtained in step c), comprises less than 1.0 mol %, such as less than 0.1 mol %, preferably less than 0.5 mol % polyhalogenated methane, as defined herein.

In accordance with the present invention, the methyl halide (MeX) stream separated in step c), is also preferably “essentially free” of polyhalogenated C₂₊ alkanes. Preferably, “essentially free of polyhalogenated C₂₊ alkanes” in this context means that the methyl halide (MeX) stream obtained in step c), comprises less than 1.0 mol %, such as less than 0.1 mol % preferably less than 0.5 mol % polyhalogenated C₂₊ alkanes, as defined herein.

In accordance with the present invention, the methyl halide (MeX) stream separated in step c), is also preferably “essentially free” of C₂₊ alkyl monohalides. Preferably, “essentially free of C₂₊ alkyl monohalides” in this context means that the methyl halide (MeX) stream obtained in step c), comprises less than 1.0 mol %, such as less than 0.1 mol % preferably less than 0.5 mol % C₂₊ alkyl monohalides, as defined herein.

In a next step, the methyl halide (MeX) stream separated in step c) of the process is reacted with a solid metal oxide (MO₍ₛ₎), under reaction conditions effective to produce metal halide (MX) and dimethyl ether (DME).

Examples of suitable metal oxides for use in the present process include but for instance metal oxides selected from the list comprising or consisting of calcium oxide (CaO), magnesium oxide (MgO), barium oxide (BaO), copper oxide (CuO), and any mixtures thereof.

Preferred examples of a solid metal oxide may be calcium oxide (CaO), magnesium oxide (MgO) or a mixture thereof, preferably the metal oxide is a mixture of calcium oxide (CaO) and magnesium oxide (MgO).

The term “solid” as used herein intends to refer to a metal oxide provided on a solid support, such as a silica or an alumina support, more preferably a silica support. Preferably, the solid support is impregnated with the metal oxide. Preferably, the solid support is impregnated with a solution of the metal oxide, after which the impregnated solid support is dried.

Step d) of the process of the invention is preferably carried at a temperature of at least 50° C., preferably at least 70° C., preferably at least 80° C., preferably at least 90° C., preferably at least

100° C., preferably at least 150° C. Step d) of the process of the invention is preferably carried out at a temperature of at most 500° C., preferably at most 450° C., preferably at most 400° C., preferably at most 350° C., preferably at most 300° C., preferably at most 250° C. For instance, preferred temperature conditions for the reaction step d) are comprised between 50° C. and 500° C., preferably between 70° C. and 450° C., preferably between 80° C. and 400° C., preferably between 90° C. and 350° C., preferably between 100° C. and 300° C., preferably between 120° C. and 250° C. In some embodiments, the temperature in step d) can be achieved by heat integration, for example with heat recovered during the exothermic reaction in step b).

Step d) of the process of the invention is preferably carried at a reaction pression of at least 1.0 bar, preferably at least 1.2 bar, preferably at least 1.5 bar, preferably at least 1.7 bar, preferably at least 2.0 bar, preferably at least 5.0 bar, preferably at least 10.0 bar. Step d) of the process of the invention is preferably carried at a reaction pression of at most 40.0 bar, preferably at most 35.0 bar, preferably at most 30.0 bar, preferably at most 25.0 bar, preferably at most 20.0 bar, preferably at most 15.0 bar, preferably at most 10.0 bar. For instance, preferred pressure conditions for the reaction step d) are comprised between 1.0 bar and 40.0 bar, preferably between 1.2 bar and 35.0 bar, preferably between 1.5 bar and 30.0 bar, preferably between 1.7 bar and 25.0 bar, preferably between 2.0 bar and 20.0 bar, preferably between 5.0 bar and 20.0 bar, preferably between 10.0 bar and 15.0 bar.

Preferably, the process of the invention, comprises a reaction in step d) of methyl halide (MeX) with the solid metal oxide (MO₍ₛ₎) for at least 0.1 min to at most 100.0 min, preferably for at least 0.2 min to at most 75.0 min, preferably for at least 0.5 min to at most 60.0 min, preferably for at least 1.0 min to at most 45.0 min, preferably for at least 2.0 min to at most 30.0 min, preferably for at least 3.0 min to at most 15.0 min, preferably for at least 4.0 min to at most 10.0 min, preferably for at least 4.0 min to at most 6.0 min.

In an example, the methyl halide is contacted in step d) with the solid metal oxide, at a temperature of between 12° and 250° C., at a pressure of between 10 bar and 15 bar, and for at least 4 min to at most 6.0 min.

It is preferred in accordance with the present invention that step d) of the process is carried out in the absence of water.

The reaction products resulting from reaction step d) mainly comprises dimethyl ether (dimethyl ether stream) and metal halide (MX). Optionally residual amounts of unreacted methane are also present in the reaction output.

A stream comprising dimethyl ether may be recovered after step d). Preferably, such recovered dimethyl ether stream comprises, based on the total amount of the stream, comprises at least 75.0 mol %, such as least 80.0 mol %, or at least 85.0 mol %, or at least 95.0 mol % of dimethyl ether. It will be noted however that the recovered methyl halide stream may still comprise certain amounts of unreacted methane.

By separating hydrogen halide from the effluent stream obtained after step b) of the process of the invention, the present process permits to subject a “purified” methyl halide stream to the ether synthesis reaction in step d) in the presence of a solid metal oxide. The separation step applied in step c) therefore permits to avoid a reaction of hydrogen halide over a solid metal oxide in step d). In this way, the present process thus avoids the conversion of hydrogen halide (HX) into metal halide (MX) and water during step d) of the process. Instead, hydrogen halide (HX) present in the effluent stream after reaction step b) is further decomposed (in step e)) into:
a substantially pure hydrogen gas stream, which is economically more interesting than water; and,
halogen reactant, which can advantageously be re-used in step b) of the process.

By separating off the hydrogen halide from the effluent stream in step c), also the production of “wet dimethyl ether” is prevented in step d); hence, avoiding the need to further dry the dimethyl ether obtained in the present process. Also, in the absence of hydrogen halides in the methyl halide stream supplied in step d), the solid metal oxide, applied in step d) of the process, remains optimally available for the conversion of methyl halide into dimethyl ether, and is not submitted to side-reactions with such hydrogen halide. In addition, by preventing the formation of metal halide (MX), as a result of an interaction with hydrogen halide, the need/frequency to regenerate metal halide formed in step d) into solid metal oxide, needed for the conversion of methyl halide into dimethyl ether, is greatly reduced.

The hydrogen halide stream obtained in step c) of the process is subject to decomposition to release the hydrogen for recovery and halogen. Said halogen reactant may be recycled to step a) and/or b) of the process, for instance added to the feed stream supplied in step a) and/or separately supplied during reaction step b).

The preferred mode of hydrogen halide decomposition is electrolytic. Thus, this step of a process of the invention preferably takes place in an electrolysis cell, preferably in a polymer electrolyte membrane cell (PEM) containing at least a proton-conductive membrane. Polymer electrolyte membrane (PEM) electrolysis is well known in the art an comprises a cell equipped with a solid polymer electrolyte (SPE) that is responsible for the conduction of protons, separation of product gases, and electrical insulation of the electrodes.

In some embodiments of the invention, step e) of the present process comprises the step of supplying an aqueous solution of hydrogen halide to an electrolysis cell (as defined herein) containing positive and negative electrodes, and decomposing said hydrogen halide electrolytically by maintaining an electrical potential from about 0.5 to 2.5 V between said electrodes.

In some embodiments of the invention, step e) of the present process comprises the step of supplying an aqueous solution of hydrogen halide to an electrolysis cell (as defined herein) containing positive and negative electrodes, and decomposing said hydrogen halide electrolytically by maintaining a current density from about 100 to 800 mA/cm² between said electrodes.

The electrical potential required to decompose the hydrogen halide solution decreases as the temperature of the aqueous solution increases. It is particularly preferred to practice the electrolytic decomposition at a temperature of from about 20 to 95° C., and preferably from about 40 to 80° C. The pressure in the electrolytic decomposition zone is maintained sufficiently high to maintain the aqueous hydrogen halide in a liquid phase. Generally, the pressure will be within a range from about 0.1 to 5 Mpa (1-50 bar).

The gaseous hydrogen (H₂) stream generated in step e) of the process comprises at least 90.0 mol %, such as at least 95.0 mol %, or at least 99.0 mol %, or at least 99.5 mol %, or at least 99.9 mol % hydrogen. Hydrogen obtained with a process according to the invention may be used in downstream applications. For instance, it may be used in ammonia synthesis, for hydrogenation purposes, for chemicals synthesis, or power generation by combustion in a gas turbine with or without additional hydrocarbon fuels, etc. Hydrogen produced can also be applied as a chemical feedstock to reduce dependence on petroleum and natural gas.

The halogen reactant (X₂) generated in step e) of the process can be re-used in step b) of the process, for instance after a suitable pre-treatment, such as a purification and/or drying.

In some preferred embodiments, the halogen reactant (X₂) obtained in step e) is purified before being returned into step b), e.g. by condensation.

In some preferred embodiments, the halogen reactant (X₂) obtained in step e) is dried before being returned into step b). Preferably, is the halogen reactant (X₂) dried with molecular sieves. Preferably, the amount of water in the halogen reactant (X₂) before the halogen reactant (X₂) is returned into step b) is at most 2000 ppm, preferably at most 1500 ppm, preferably at most 1000 ppm, preferably at most 750 ppm, preferably at most 500 ppm, wherein the ppm is expressed as the weight of water compared to the total weight of the halogen reactant.

In some embodiments the present invention, a process of the invention further comprises the step f) of regenerating the metal halide (MX), formed in step d), to metal oxide (MO) and halogen reactant (X₂).

The term “regenerating” and “converting” are used in this context as synonyms. The present process preferably comprises the recovery of metal oxide and a halogen reactant, by contacting the metal halide (MX) obtained in step d) of the process with regenerating agent capable of forming MO and halogen reactant (X₂).

A “regenerating agent” for use in step f) of the process may include oxygen or air. For example, the present process preferably comprises the recovery of metal oxide and a halogen reactant, by contacting the metal halide (MX) obtained in step d) of the process with oxygen or with air, thereby forming MO and halogen reactant (X₂). The particular amount of air or oxygen may depend on particle size, loading of metal oxide on the solid support and selected regeneration temperature. Preferably, an excess of air or oxygen is used.

In accordance with the invention, it is preferred that step f) of the process is carried out at a temperature of at least 150° C., preferably at least 200° C., preferably at least 250° C., preferably at least 300° C., preferably at least 350° C., preferably at least 400° C. It is preferred that step f) of the process is carried out at a temperature of at most 1600° C., preferably at most 1400° C., preferably at most 1200° C., preferably at most 1000° C., preferably at most 800° C., preferably at most 600° C. For instance, the temperature during step f) of the process is from at least 150° C. to at most 1600° C., preferably from at least 200° C. to at most 1400° C., preferably from at least 250° C. to at most 1200° C., preferably from at least 300° C. to at most 1000° C., preferably from at least 350° C. to at most 800° C., preferably a from at least 400° C. to at most 600° C. Preferably, the temperature during step f) may be maintained by the exothermic reaction in step f) and optionally by the provision of steam.

In accordance with the invention, it is preferred that step f) of the process is carried out at a pressure during step f) of at least 0.5 bar, preferably at least 0.8 bar, preferably at least 0.9 bar, preferably at least 1.0 bar, preferably at least 1.1 bar, preferably at least 5.0 bar, preferably at least 10.0 bar. It is preferred that step f) of the process is carried out at a pressure during step f) of at most 50.0 bar, preferably at most 45.0 bar, preferably at most 40.0 bar, preferably at most 35.0 bar, preferably at most 30.0 bar, preferably at most 25.0 bar, preferably at most 20.0 bar. For instance, the reaction pressure during step f) is at least 0.5 bar to at most 50.0 bar, preferably at least 0.8 bar to at most 45.0 bar, preferably at least 0.9 bar to at most 40.0 bar, preferably at least 1.0 bar to at most 35.0 bar, preferably at least 1.1 bar to at most 30.0 bar, preferably at least 5.0 bar to at most 25.0 bar, preferably at least 10.0 bar to at most 20.0 bar, preferably at least 10.0 bar to at most 15.0 bar.

In an example, the temperature during step b) is at least 300° C. to at most 600° C., and the pressure during step b) is at least 5.0 bar to at most 30.0 bar. In another example, the temperature during step b) is comprised between 380° C. and 520° C., and the pressure during step b) is comprised between 10.0 bar and 20.0 bar. In an example, the temperature during step d) is at least 100° C. to at most 300° C., and the pressure during step d) is at least 2.0 bar to at most 20.0 bar. In another example, the temperature during step d) is comprised between 120° C. and 250° C., and the pressure during step d) is comprised between 10.0 bar and 15.0 bar. In an example, the temperature during step f) is at least 300° C. to at most 1000° C., and the pressure during step f) is at least 1.1 bar to at most 30.0 bar. In another example, the temperature during step f) is comprised between 400° C. and 600° C., and the pressure during step f) is comprised between 10.0 bar and 15.0 bar.

The metal halide formed in step d) can in certain instances be separated from the metal oxide for performing step f. During step d), it may also happen that the amount of solid metal oxide is depleted whereas the amount of metal halide on solid support is increased, thereby forming a mixture of metal oxide and metal halide on the solid support. In such cases, regeneration step f) may be performed on the mixture of metal oxide and metal halide formed on the solid support during step d).

The regeneration of the metal halide (MX), formed in step d), into a metal oxide can not be performed simultaneously with the conversion of methyl halide into dimethyl ether during this step. Therefore, in some embodiments, a portion of the solid support, comprising e.g. a mixture of metal halide and metal oxide may be separated or isolated from the reactor or reaction zone to be regenerated in step f), while another portion is used for carrying out step d). For instance, multiple beds of solid metal oxide can be used, so that one bed can be used for the formation of dimethyl ether in step d) while the other bed is being regenerated in a step g). In such embodiment, reactor beds may be alternatively subjected to a regeneration in accordance with step f). The metal oxide obtained by the regeneration step can be re-used in the process as solid metal oxide in step d).

Regeneration results in the formation of a gaseous effluent, comprising halogen reactant, oxygen, and optionally nitrogen. Preferably, the halogen reactant (X₂) is isolated from the gaseous effluent, preferably by condensation of the halogen reactant (X₂) from the gaseous effluent. The obtained halogen reactant can also be re-used in step b) of the process, for instance after a suitable pre-treatment, such as a purification and/or drying.

In some preferred embodiments, the halogen reactant (X₂) obtained in step f) is purified before being returned into step b), e.g. by condensation.

In some preferred embodiments, the halogen reactant (X₂) obtained in step f) is dried before being returned into step b). Preferably, is the halogen reactant (X₂) dried with molecular sieves. Preferably, the amount of water in the halogen reactant (X₂) before the halogen reactant (X₂) is returned into step b) is at most 2000 ppm, preferably at most 1500 ppm, preferably at most 1000 ppm, preferably at most 750 ppm, preferably at most 500 ppm, wherein the ppm is expressed as the weight of water compared to the total weight of the halogen reactant.

In certain embodiments of a process according to the invention, polyhalogenated methane is formed during step b), and may be separated during step c), as indicated above. In certain embodiments, a process of the invention therefore further comprises the step of converting said polyhalogenated methane, e.g. separated in step c), to methanol, preferably by hydrogenation of polyhalogenated methane to form methyl halide, followed by contacting methyl halide with a solid metal hydroxide (MOH₍ₛ₎), under reaction conditions effective to produce the corresponding methanol.

Examples of suitable metal hydroxides for use in the present process include but for instance metal hydroxides selected from the list comprising or consisting of potassium hydroxide (KOH), sodium hydroxide (NaOH), barium hydroxide (Ba(OH)₂), caesium hydroxide (CsOH), strontium hydroxide (Sr(OH)₂), calcium hydroxide (Ca(OH)₂), lithium hydroxide (LiOH) rubidium hydroxide (RbOH), magnesium hydroxide (Mg(OH)₂), and any mixtures thereof. Preferred examples of a solid metal hydroxide may be alkali hydroxides, such as preferably potassium hydroxide (KOH), lithium hydroxide (LiOH), sodium hydroxide (NaOH), or any mixtures thereof. A particularly preferred example is potassium hydroxide (KOH).

The term “solid” as used herein intends to refer to a metal hydroxide provided on a solid support, such as a silica or an alumina support, more preferably a silica support. Preferably, the solid support is impregnated with the metal hydroxide. Preferably, the solid support is impregnated with a solution of the metal hydroxide, after which the impregnated solid support is dried.

Examples of suitable reaction conditions include for instance contacting the methyl halide with the solid metal hydroxide, at a temperature of between 12° and 250° C., at a pressure of between 10 bar and 15 bar, and for at least 4 min to at most 6.0 min.

In certain embodiments of a process according to the invention, polyhalogenated methane is formed during step b), and may be separated during step c), as indicated above. In certain embodiments, a process of the invention therefore further comprises the step of converting said polyhalogenated methane, e.g. separated in step c), to DME, preferably by hydrogenation of polyhalogenated methane to form methyl halide, followed by contacting methyl halide with a solid metal oxide, preferably as defined herein, under reaction conditions effective to produce DME, preferably under reaction conditions as defined herein.

In certain embodiments of the present invention, unreacted methane may remain present in the effluent stream obtained in step b) of the process. In certain embodiments of the present invention, unreacted methane may remain present in the methyl-halide stream obtained in step c) of the process. In certain embodiments of the present invention, unreacted methane may remain present in the stream of dimethyl ether obtained in step d) of the present process. Remaining unreacted methane may be removed from said respected streams, e.g. by distillation. In certain preferred embodiments of the invention, such removed unreacted methane can be recycled or fed into step a) and/or step b) of the process.

The invention provides a system for producing dimethyl ether and hydrogen from methane, comprising;
a halogenation reactor (1), configured to react a gaseous feed stream comprising methane with at least one halogen reactant (X₂) into an effluent stream comprising methyl halide (MeX), and hydrogen halide (HX), and optionally unreacted methane,
a separation system (2), which is fluidly connected to said halogenation reactor (1) and configured to receive an effluent stream from said halogenation reactor (1) and configured to separate a hydrogen halide (HX) stream and a methyl halide (MeX) stream from said effluent stream;
a dimethyl ether synthesis reactor (3), which is fluidly connected to said separation system (2), and configured to receive a methyl halide stream from said separation system (2) and to react said methyl halide stream with a solid metal oxide into metal halide (MX) and dimethyl ether (DME); and
an electrolysis unit (4) comprising at least one electrolysis cell and a power source for supplying current to said electrolysis cell, which is fluidly connected to said separation system (2), and configured to receive a hydrogen halide stream and to decompose said hydrogen halide stream into a gaseous hydrogen stream and a stream comprising halogen reactant.

As used herein, the term “fluidly connected” or “in fluid connection” are used herein as synonyms and intend to refer to a connection between two components (or units or devices or the like) of the system as given herein, so that trough said connection a fluid can flow from one component (e.g. unit, device, reactor, etc) to the other. Preferably two fluidly connected components are connected so that an outlet of the first component is connected by a pipe, a conduit or a series of pipes or conduits, to an inlet of the second component. Other components, such as separators, dryers, condensers and the like, may be fluidly connected between two fluidly connected components.

The terms “means for supplying” or “supply system” are used herein as synonyms and refer to suitable systems allowing the supply of a stream, or reaction product, or reactant, and includes for instance pipes, conduits, supply lines, inlet lines, connecting lines, and the like.

The term “means for recovering” or “recovery system” are used herein as synonyms and refer to suitable systems allowing the recovery of a stream or reaction product or reactant, and includes for instance pipes, recovery lines, outlet lines, connecting lines, and the like. “Means for recovering” or “recovery system” may comprise separation means.

The term “means for transferring” or “transfer system” are also used herein as synonyms and refer to suitable systems allowing the transfer (transport) of a stream or reaction product or reactant, e.g. from one unit or reactor to another unit or reactor, and includes for instance pipes, conduits, recovery lines, outlet lines, connecting lines, and the like.

A halogenation reactor (1) for use in a system of the invention, may be any type of reactor suitable for allowing a gaseous feed stream comprising methane to react with a halogen reactant under suitable conditions for yielding an effluent stream comprising methyl halide (MeX), and hydrogen halide (HX).

In certain preferred embodiments, a separation system (2) for use in a system of the invention comprises or consists of a quench column. In preferred embodiments of the invention, the separation system, preferably the quench column, is equipped with an inlet system, configured to supply an absorbent, as defined herein, to said system or column.

An dimethyl ether synthesis reactor (3) for use in a system of the invention may be any type of reactor suitable for housing a solid metal oxide, as defined herein, and allowing a (gaseous) methyl halide stream to react with said solid metal oxide under suitable conditions for producing a metal halide (MX) and dimethyl ether (DME).

In an example, said dimethyl ether synthesis reactor (3) is a swing-bed reactor. As used herein, the term “swing-bed reactor” refers to a reactor, which comprises at least two beds, for accommodating solid metal oxide, the beds are organised so that one bed can be regenerated, while the other one can be used to covert metal halide into dimethyl ether.

In another example, the dimethyl ether synthesis reactor (3) comprises at least one, preferably at least two fluidized beds. Especially when there are at least two fluidised beds, one bed can be taken out of use and be regenerated while the other bed is still used for converting methyl halide into dimethyl ether.

An electrolysis unit (4) for use in the present invention comprises at least one electrolysis cell and a power source for supplying current to said electrolysis cell. A particularly suitable electrolysis cell is a polymer electrolyte membrane cell (PEM) containing at least a proton-conductive membrane. One of the largest advantages to PEM electrolysis is its ability to operate at high current densities. This can result in reduced operational costs. The polymer electrolyte allows a PEM electrolytic cell to operate with a very thin membrane (˜100-200 μm) while still allowing high pressures, resulting in low ohmic losses, primarily caused by the conduction of protons across the membrane (0.1 S/cm) and a compressed hydrogen output.

The polymer electrolyte membrane, due to its solid structure, advantageously gives very high product gas purity.

A system according to the present invention may also comprise
a feed stream supply system (5) for supplying a gaseous feed stream comprising methane to said halogenation reactor (1); and
a halogen supply system (6) for supplying a halogen reactant to said halogenation reactor (1).

Such systems for supplying reactants to the halogenation reactor may include inlet lines (conduits), optionally provided with controlling means for controlling flow rate of the reactant streams to the reaction zone. In preferred embodiments of the invention separate means (separate inlet lines or conduits) may be provided for each of the reactants in the process, i.e. separately for the gaseous feed stream and for the halogen reactant.

A system according to the present invention may also comprise an effluent recovery system (10), e.g. a conduit or pipe, configured to recover an effluent stream from said halogenation reactor (1), and adapt to feed said recovered effluent stream to the downstream separation system (2).

A system according to the present invention may also comprise
a hydrogen halide transfer system (12), configured to recover a hydrogen halide stream from said separation system (2), and to supply said recovered hydrogen halide stream to the electrolysis unit (4); and
a methyl halide transfer system (11), configured to recover a methyl halide stream from said separation system (2), and to supply said recovered methyl halide stream to the dimethyl ether synthesis reactor (3).

Such systems for supplying or recovering reactants to and from the separation system, may include inlet lines (conduits, pipes, lines), optionally provided with controlling means, e.g., for controlling flow rate of the reactant streams to the reaction zone.

A system according to the present invention may also comprise an absorbent supply line (17), for supplying an aqueous absorbent, preferably an aqueous hydrogen halide (HX) solution, to the separation system (2). The absorbent supply line is for instance an inlet line, for supplying an absorbent, e.g. an aqueous solution of hydrogen halide, to said separation zone.

A system in accordance with the present invention may also comprise systems for separately recovering a gaseous hydrogen stream and a stream comprising halogen reactant from the electrolysis unit. Such systems comprise in particular,
a hydrogen recovery system (8), configured to recover a gaseous hydrogen stream from said electrolysis unit (4);
a halogen recovery system (9), configured to recover halogen reactant from said electrolysis unit (4);

Such systems for supplying or recovering reactants to and from the separation system, may include inlet lines (conduits, pipes, lines), optionally provided with controlling means, e.g.; for controlling flow rate of the reactant streams to the reaction zone.

Optionally, the system of the invention may be provided with means (20) (e.g. a transfer line or conduit) for returning the stream of halogen reactant recovered from said electrolysis unit, or least a part thereof, to the halogenation reactor. This allows for the re-use of halogen reactant in the system.

In certain preferred embodiments, the means (20) for returning the stream of halogen reactant, or a part thereof, recovered from said electrolysis unit (4) to said halogenation reactor (1) comprise one or more connecting lines (connecting pipes).

In certain embodiments, said one or more connecting lines for returning the stream of halogen reactant to said halogenation reactor may be provided with one of more dryers (e.g. molecular sieves), suitable for drying the stream of halogen reactant prior to feeding said stream to said halogenation reactor.

In certain embodiments, said one or more connecting lines for returning the stream of halogen reactant to said halogenation reactor may be provided with one of more purification means, such as e.g. one or more condensers, suitable for purifying the stream of halogen reactant prior to feeding said stream to said halogenation reactor.

Furthermore, the system of the invention comprises a dimethyl ether recovery system (7), configured to recover a stream comprising dimethyl ether from said dimethyl ether synthesis reactor (3).

In certain embodiments of the present invention, polyhalogenated alkanes may be formed in step b) of the process. Such polyhalogenated alkanes can for instance comprise methyl dihalide (CH₂X₂). Therefore, a system according to the present invention may comprise in certain embodiments a polyhalogenated alkanes removal unit, configured to receive effluent stream from said halogenation reactor (1) and to remove polyhalogenated alkanes from said effluent stream, thereby producing a polyhalogenated alkanes stream and a methyl halide stream.

Preferably, such polyhalogenated alkanes removal unit is provided downstream of the halogenation reactor (1) and upstream of the separation system (2), and is connected, preferably fluidly connected, to the halogenation reactor (1) and to the separation system (2), e.g. by means of connecting lines (15, 16).

The polyhalogenated alkanes removal unit may for instance comprises:
a separation system (13), such as a separation (e.g. distillation) column, configured to receive an effluent stream from said halogenation reactor (1) and to separate from said effluent stream a polyhalogenated alkanes stream,
a supply system (15), such as a supply line or pipe, configured for supplying an effluent stream from said halogenation reactor (1) to said separation column, and
a polyhalogenated alkanes recovery system (14), such as an outlet line or pipe, configured for recovering a polyhalogenated alkanes stream from said separation system (13).

The polyhalogenated alkanes removal unit preferably also comprises means, such as a transfer line or pipe, for recovering a product stream (e.g., a product stream comprising methyl halide and hydrogen halide), from which polyhalogenated alkanes as defined herein have been removed. Preferably such means are configured to transfer the recovered product stream to a downstream separation system, for further separation of the product stream, e.g. in a separate methyl halide stream and a separate hydrogen halide stream.

In certain embodiments, the polyhalogenated alkanes removal unit is able to separate polyhalogenated alkanes from the effluent stream into a polyhalogenated methane stream and a polyhalogenated C₂₊ alkane stream. In certain embodiments, the polyhalogenated alkanes removal unit comprises a polyhalogenated methane outlet and a polyhalogenated C₂₊ alkanes outlet.

In certain embodiments of the present invention, C₂₊ alkyl monohalides, as defined herein, may be formed in step b) of the process. Therefore, in certain embodiments a system according to the present invention may also comprise a C₂₊ alkyl monohalide removal unit, configured to receive effluent stream from said halogenation reactor (1) and to remove C₂+ alkyl monohalides from said effluent stream.

In certain embodiments, such C₂₊ alkyl monohalide removal unit is provided downstream of the halogenation reactor (1) and upstream of the separation system (2), and is connected, preferably fluidly connected, to the halogenation reactor (1) and to the separation system (2), e.g. by means of connecting lines.

In certain embodiments, a C₂₊ alkyl monohalide removal unit may for instance comprises
a separation system, such as a separation (e.g. a distillation) column, configured to receive an effluent stream from said halogenation reactor (1) and to separate from said effluent stream a C₂₊ alkyl monohalide stream,
a supply system, such as a supply line or pipe, configured for supplying an effluent stream from said halogenation reactor (1) to said separation system, and
a C₂₊ alkyl monohalide recovery system, such as an outlet line or pipe, configured for recovering a C₂₊ alkyl monohalide stream from said C₂₊ alkyl separation system.

A C₂₊ alkyl monohalide removal unit preferably also comprises means, such as a transfer line or pipe, for recovering a product stream (e.g., a product stream comprising methyl halide and hydrogen halide), from which the C₂₊ alkyl monohalides have been removed. Preferably such means are configured to transfer the recovered product stream to a downstream separation system, for further separation of the product stream, e.g. in a separate methyl halide stream and a separate hydrogen halide stream.

In certain embodiments of the invention, the present system further comprises
a supply line (18), configured to supply, to the dimethyl ether synthesis reactor (3), a regenerating agent, as defined herein, capable of converting the metal halide (MX), formed in said dimethyl ether synthesis reactor, into metal oxide (MO) and halogen reactant (X₂);
an outlet line (19), configured to remove, from the dimethyl ether synthesis reactor, an effluent stream comprising halogen reagent (X₂) generated during said conversion reaction.

By providing the above-named elements, the present system makes it possible to regenerate (recover) solid metal oxide within inside the same system. This avoids the need for a delivery system for supplying fresh solid metal oxide to the system and process, and hence also improves process sustainability and efficiency.

Optionally, the system of the invention may be further provided with separation means for separating halogen reagent from the effluent stream comprising halogen reagent (X₂) generated during said conversion (regeneration) reaction. Suitable separation means for separating said halogen reagent from said effluent stream may include but are not limited to one or more condensers.

In certain preferred embodiments, the system further provides means (19, 20), for returning a stream of halogen reactant recovered from said dimethyl ether synthesis reactor (3) to said halogenation reactor (1). In certain preferred embodiments, the means (19) for returning halogen reactant, recovered from said dimethyl ether synthesis reactor (3) to said halogenation reactor (1) comprise one or more connecting lines (connecting pipes). In certain embodiments, said one or more connecting lines for returning the stream of halogen reactant to said halogenation reactor may be provided with one of more dryers (e.g. molecular sieves or the like), suitable for drying the stream of halogen reactant prior to feeding said stream to said halogenation reactor. In certain embodiments, said one or more connecting lines for returning the stream of halogen reactant to said halogenation reactor may be provided with one of more purification means, such as e.g. one or more condensers, suitable for purifying the stream of halogen reactant prior to feeding said stream to said halogenation reactor.

In certain embodiments of the present invention, unreacted methane may remain present in the effluent stream obtained in step b) of the process. In certain embodiments of the present invention, unreacted methane may remain present in the methyl-halide stream obtained in step c) of the process. In certain embodiments of the present invention, unreacted methane may remain present in the stream of dimethyl ether obtained in step d) and/or f) of the present process. Remaining unreacted methane may be removed from said respected stream, e.g. by distillation. Therefore, the present system further provides means, such as distillation columns or the like, for removing, preferably distilling, unreacted methane, from above mentioned streams. In a particularly preferred embodiment of the present invention, a system is provided in which a distillation column is provided in fluid connection with the dimethyl ether recovery system (7).

The following examples serve to merely illustrate the invention and should not be construed as limiting its scope in any way. While the invention has been shown in only some of its forms, it should be apparent to those skilled in the art that it is not so limited, but is susceptible to various changes and modifications without departing from the scope of the invention.

### EXAMPLES

### Example 1

The following example demonstrates an embodiment of a process according to the invention, and a system for carrying out such embodiment according to the invention, with reference to FIG. 1.

The system depicted in FIG. 1 comprises a halogenation reactor (1), a separation system (2), an dimethyl ether synthesis reactor (3) and an electrolytic unit (4).

According to this embodiment, a gaseous feed stream comprising methane is introduced into the halogenation reactor (1) via a feed stream supply line (5). A halogen reactant (e.g. Br₂) is also introduced into this halogenation reactor (1), through a separate halogen reactant supply line (6). The gaseous feed stream comprising methane reacts with the halogen reactant (X₂) in the halogenation reactor, yielding an effluent stream comprising methyl halide (MeX) and hydrogen halide (HX). Optionally unreacted methane remains present in this effluent stream.

The obtained effluent stream exits the halogenation reactor (1) through a suitable recovery line (10) and is fed via said line into the separation system (2), which is provided downstream the halogenation reactor. In the separation system, the effluent stream is further separated into a hydrogen halide (HX) stream, which is recovered from the separation unit and fed, via a suitable connecting line (12), to an electrolysis unit (4); and, a methyl halide (MeX) stream, which is recovered from the separation system, and fed via a suitable connecting line (11) to the dimethyl ether synthesis reactor (3).

In the dimethyl ether synthesis reactor (3) the methyl halide (MeX) stream is reacted with a solid metal oxide, whereby dimethyl ether (DME) is formed. A stream comprising dimethyl ether can be recovered from the dimethyl ether synthesis reactor (3) through a dimethyl ether recovery line (7).

In the electrolysis unit (4), the hydrogen halide (HX) stream, issued from the separation system, is further decomposed into stream of hydrogen gas (H₂), which is removed from the electrolysis unit via outlet line (8); and a stream of halogen reactant (X₂), which is removed from the electrolysis unit via outlet line (9).

### Example 2

The following example demonstrates another embodiment of a process according to the invention, and a system for carrying out such embodiment according to the invention, with reference to FIG. 2.

The system depicted in FIG. 1 comprises a halogenation reactor (1), a separation system (2), a dimethyl ether synthesis reactor (3), an electrolytic unit (4), and a polyhalogenated alkanes removal unit (13).

A gaseous feed stream comprising methane containing about 100 mol. % (CH₄) was introduced into a halogenation reactor (1) via feed stream supply line (5) at a flow rate of about 1604 kg/hr. The halogen reactant (X₂), in this case bromine (Br₂), was introduced to the halogenation reactor (1) through the halogen reactant supply line (6) at a rate of about 5274 kg/hr. The halogenation reactor (1) was maintained at a temperature of about 300-600° C. and a pressure from 5 to 30 bar.

An effluent stream comprising unreacted methane (CH₄), hydrogen halide (HX), methyl halide (MeX), and polyhalogenated alkanes (e.g. CH₂X₂) was recovered from the halogenation reactor (1), via outlet (15) and fed to a polyhalogenated alkanes removal unit comprising a separation column (13). This separation column separates polyhalogenated alkanes (e.g. CH₂X₂), e.g. CH₂Br₂, from the effluent stream and removes polyhalogenated alkanes (e.g. CH₂X₂) from the system via a polyhalogenated alkanes outlet line (14) at rate of around 860 kg/hr.

The residual gaseous stream which is substantially free of polyhalogenated alkanes (like CH₂X₂), is recovered from the polyhalogenated alkanes removal unit (13), and is then fed, via a connecting line (16), into to the separation system (2) comprising a quench column. In the quench column, the residual gas stream is contacted with an aqueous absorbent, in particular in this example a 26.7 wt. % aqueous HBr solution, with a flow rate of around 13800 kg/hr. The absorbent captures gaseous hydrogen halide (HX) from the effluent stream, and the resulting solution leaves the separation system (2) as a liquid stream through a hydrogen halide outlet line (12).

The remaining gaseous stream substantially comprises methyl halide (MeX), and optionally some unreacted methane (CH₄), leaves the separation system (2) via outlet line (17), and is fed to a DME synthesis reactor (3). In the present example, the DME synthesis reactor (3) is a swing-bed reactor filled with calcium-magnesium oxide deposited on porous y-Alumina (MO₍ₛ₎). The dimethyl ether synthesis reactor (3) is maintained at a temperature of about 100-300° C. and a pressure from 2 to 30 bar.

In the dimethyl ether synthesis reactor (3) methyl halide (MeX) is converted into dimethyl ether (DME), which is recovered together with optional unreacted methane (CH₄) through dimethyl ether outlet (7). Dimethyl ether is produced at a rate of around 740 kg/hr and can be separated from unreacted methane by techniques known in the art, e.g. distillation with a distillation column.

The hydrogen halide (HX) liquid stream leaving the separation system via the hydrogen halide outlet line (12) is sent to the electrolysis unit (4), where the hydrogen halide (HX) is decomposed into hydrogen gas (H₂) and halogen reactant (X₂), in this example Br₂. Produced hydrogen gas (H₂) may be recovered through a hydrogen gas outlet line (8) and is optionally dried. The produced halogen reactant (X₂) may also be recovered through an outlet line (9), optionally dried, and returned to the halogenation reactor (1) for re-use.

In accordance with the present process, is may happen that the (bed of the) dimethyl ether synthesis reactor becomes depleted of reactant, i.e. in this example calcium-magnesium oxide (MOs), i.e. when too much (MX(s)) is formed in the bed. In that case, the reactor bed may be regenerated, by treating it with a regenerating agent (18), comprising air (comprising O₂ and N₂), at temperatures of 300 to 1000° C. and a pressure from 1.1 to 30 bar. This regeneration treatment results in the formation of metal oxide (MO) and halogen reactant (X₂).

Gaseous effluent generated during such regeneration process, comprises nitrogen (N₂), oxygen (O₂) and halogen reagent (X₂), and can leave the hydrogenation reactor (3) via effluent outlet (19). The halogen reagent (X₂) may then be separated from said effluent, e.g. by condensation, dried, and sent back to the halogenation reactor (1) for re-use in the system.

### Example 3

This example illustrates a halogenation step as may be applied in a process of the invention.

In this example, the experimental setup comprised a quartz reactor and a glass syringe for liquid bromine supply (Sigma Aldrich). A methane (5.0, Praxair) supply with Bronkhorst mass-flow controller was created.

The feed rate was maintained to have a residence time for reactants in the heated zone of the reactor of around 0.01 s and the molar ratio of CH₄/Br₂ of about 3/1. The heated zone of the reactor was kept at a temperature of about 370° C. and pressure of about 3 bar.

The effluent of the reactor was then first passed through a cold trap (10° C.) to condense CH₂Br₂, produced during the reaction, and trace amounts of unreacted bromine.

In this experiment, the effluent of the reactor was passed through an aqueous caustic bubbler to remove major amount of HBr prior to sending the remaining stream for analysis to an Agilent GC with SilicaPlot column and FID detector.

The conversion of methane into methyl bromide, with side production of CH₂Br₂, was calculated on the basis of GC data, using the following formula:

$$x ( CH 4 ) = \frac{n ( CH {4}_{feed} ) - n ( CH {4}_{effluent} )}{n ( CH {4}_{feed} )}$$

$$s ( CH 3 Br ) = \frac{n ( CH 3 {Br}_{effluent} )}{n ( CH {4}_{feed} ) - n ( CH {4}_{effluent} )}$$

$$s ( CH 2 Br 2 ) = \frac{n ( CH 2 Br {2}_{effluent} )}{n ( CH {4}_{feed} ) - n ( CH {4}_{effluent} )}$$

x(CH₄)=29%,
s(CH₃Br)=89%,
s(CH₂Br₂)=9%.
wherein n=molar quantity, x=conversion and s=selectivity

### Example 4

The following example illustrates an ether synthesis reaction step as may be applied in a process of the invention.

In this example, the experimental setup comprised a vertical quartz reactor (20 mm ID) connected to a GC was used. Methyl bromide (>99.9%, Mebrom) was fed through a piston pump. The reactor was packed with 240 g of mixed calcium-magnesium oxide sample deposited on porous γ-Alumina, pre-calcined before the test. The temperature of the reactor was kept at 120° C. and 5.5 barA during the entire experiment. Flowrates of CH₃Br of 25 g/h and of N₂ of 280 mL/min were used during the experiment. The conversion of CH₃Br into dimethyl ether appeared to be stable during 4 h of the run.

The conversion of methyl bromide into dimethyl ether was calculated on the basis of GC data, using the following formula

$$x ( CH 3 Br ) = \frac{n ( CH 3 {Br}_{feed} ) - n ( CH 3 {Br}_{effluent} )}{n ( CH 3 {Br}_{feed} )}$$

$$s ( D M E ) = \frac{n ( D M {E}_{effluent} )}{n ( CH 3 {Br}_{feed} ) - n ( CH 3 {Br}_{effluent} )}$$

With x (CH₃Br) being higher 45%, and s (DME)=83%; wherein n=molar quantity, x=conversion and s=selectivity

1. A process for producing dimethyl ether (DME) and hydrogen (H₂) from methane, comprising the steps of:
a) providing a gaseous feed stream comprising methane;
b) reacting said gaseous feed stream with at least one halogen reactant (X₂), under reaction conditions effective to produce an effluent stream comprising methyl halide (MeX), and hydrogen halide (HX), optionally polyhalogenated alkanes, and optionally unreacted methane;
c) separating from the effluent stream obtained in step b):
(i) a methyl halide (MeX) stream, optionally comprising unreacted methane; and,
(ii) an aqueous hydrogen halide (HX) stream or a hydrogen halide (HX) stream;
d) reacting the methyl halide (MeX) stream separated in step c) with a solid metal oxide (MO₍ₛ₎), under reaction conditions effective to produce metal halide (MX) and dimethyl ether (DME); and,
e) decomposing by means of electrolysis said aqueous hydrogen halide (HX) stream or said hydrogen halide (HX) stream separated in step c) under conditions effective to produce a gaseous hydrogen (H₂) stream and a stream comprising halogen reactant (X₂);
wherein in step c) the hydrogen halide (HX) stream is separated from the effluent stream by contacting the effluent stream comprising said hydrogen halide with an aqueous absorbent, or by distillation, thereby forming respectively an aqueous hydrogen halide (HX) stream or a hydrogen halide (HX) stream.

2. The process according to claim 1, wherein the process further comprises the step of separating said polyhalogenated alkanes formed in step b) from said effluent stream.

3. (canceled)

4. (canceled)

5. The process according to claim 1, wherein said polyhalogenated alkanes comprise polyhalogenated methane and optionally polyhalogenated C₂₊ alkanes.

6. The process according to claim 1, wherein said halogen reactant is selected form the group consisting of bromine (Br₂), chlorine (Cl₂), fluorine (F₂), iodine (I₂), and astatine (At₂).

7. (canceled)

8. The process according to claim 1, wherein the aqueous absorbent used in step c) is an aqueous hydrogen halide (HX) solution.

9. The process according to claim 1, wherein C₂₊ alkyl monohalides are formed during step b).

10. The process according to claim 1, wherein the process comprises the step of separating C₂₊ alkyl monohalides formed in step b) of the process from said effluent stream.

11. The process according to claim 10, wherein the process comprises separating said C₂₊ alkyl monohalides formed in step b) from said effluent stream prior to separating said methyl halide (MeX) stream and said hydrogen halide stream from said effluent stream.

12. (canceled)

13. (canceled)

14. (canceled)

15. (canceled)

16. (canceled)

17. The process according to claim 1, wherein the solid metal oxide (MO)(s)) is a metal oxide provided on a solid support.

18. The process according to claim 17, wherein said solid support is a silica or an alumina support.

19. The process according to claim 1, wherein the process further comprises the step of:
f) regenerating the metal halide (MX) to metal oxide (MO) and halogen reactant (X₂), thereby forming metal oxide (MO) and halogen reactant (X₂).

20. (canceled)

21. The process according to claim 1, further comprising the step of returning the halogen reactant (X₂) obtained in step e) and/or step f), to step b) of the process.

22. (canceled)

23. (canceled)

24. The process according to claim 1, wherein the process further comprises the step of removing unreacted methane from the dimethyl ether produced in step d).

25. The process of claim 24, wherein the step of removing unreacted methane from the dimethyl ether produced in step d) is distilling unreacted methane from the dimethyl ether produced in step d).

26. The process of claim 24, wherein the process further comprises the steps of recycling or feeding said removed unreacted methane into step a) and/or step b) of the process.

27. System for producing dimethyl ether (DME) and hydrogen (H₂) from methane, comprising:
a halogenation reactor (1), configured to react a gaseous feed stream comprising methane with at least one halogen reactant (X₂) into an effluent stream comprising methyl halide (MeX), and hydrogen halide (HX), and optionally unreacted methane,
a separation system (2), which is fluidly connected to said halogenation reactor (1) and configured to receive an effluent stream from said halogenation reactor (1) and configured to separate a hydrogen halide (HX) stream and a methyl halide (MeX) stream from said effluent stream;
a dimethyl ether synthesis reactor (3), which is fluidly connected to said separation system (2), and configured to receive a methyl halide (MeX) stream from said separation system (2) and to react said methyl halide (MeX) stream with a solid metal oxide (MO₍ₛ₎) into metal halide (MX) and dimethyl ether (DME); and
an electrolysis unit (4) comprising at least one electrolysis cell and a power source for supplying current to said electrolysis cell, which is fluidly connected to said separation system (2), and configured to receive a hydrogen halide (HX) stream and to decompose said hydrogen halide (HX) stream into a gaseous hydrogen (H₂) stream and a stream comprising halogen reactant (X₂);
a feed stream supply system (5) for supplying a gaseous feed stream comprising methane to said halogenation reactor (1);
a halogen supply system (6) for supplying a halogen reactant (X₂) to said halogenation reactor (1);
an effluent recovery system (10), configured to recover an effluent stream from said halogenation reactor (1), and for feeding said recovered effluent stream to said separation system (2);
a dimethyl ether recovery system (7), configured to recover a stream comprising dimethyl ether (DME) from said dimethyl ether synthesis reactor (3);
a hydrogen recovery system (8), configured to recover a gaseous hydrogen (H₂) stream from said electrolysis unit (4);
a halogen recovery system (9), configured to recover halogen reactant (X₂) from said electrolysis unit (4);
a hydrogen halide transfer system (12), configured to recover a hydrogen halide (HX) stream from said separation system (2), and to supply said recovered hydrogen halide (HX) stream to the electrolysis unit (4);
a methyl halide transfer system (11), configured to recover a methyl halide (MeX) stream from said separation system (2), and to supply said recovered methyl halide (MeX) stream to the dimethyl ether synthesis reactor (3)

28. System according to claim 27, wherein the system further comprises an absorbent supply line (17) for supplying an aqueous absorbent to said separation system (2).

29. System according to claim 28, wherein said aqueous absorbent is an aqueous hydrogen halide (HX) solution.

30. System according to claim 28, wherein the system further comprises a polyhalogenated alkanes removal unit, configured to receive effluent stream from said halogenation reactor (1) and to remove polyhalogenated alkanes from said effluent stream.

31. System according to claim 28, wherein the system further comprises a C₂₊ alkyl monohalide removal unit, configured to receive effluent stream from said halogenation reactor (1) and to remove C₂₊ alkyl monohalides from said effluent stream.
